SEQUENCE LISTING (1 sequence)

SEQ ID NO: 1 - protein, 135 aa, Artificial Sequence
  Synthetic polypeptide
GSDLDKKLLEAARAGQDDEVRILMANGADVNARDSTGWTPLHLAAPWGHPEIVEVLLKNGADVNAADFQGWTPLHLAAAVGHLEIVEVLLKYGADVNAQDKFGKTAFDISIDNGNEDLAEILQKAAGGGSGGGSC

(19) United States
(12) Patent Application Publication
(10) Pub. No.: US 2022/0331258 A1
(43) Pub. Date: Oct. 20, 2022

(54) Poly-Dioxanone Multi-Block Copolymer for Ocular Protein Delivery

(71) Applicant: Allergan Sales, LLC, Irvine, CA (US)

(72) Inventors: Patrick Hughes, Aliso Viejo, CA (US); Hongwen Rivers, San Marcos, CA (US); Jie Shen, Irvine, CA (US); Johan Zuidema, Groningen (NL); Henk Haitjema, Groningen (NL); Christine Hiemstra, Groningen (NL); Rob Steendam, Groningen (NL)

(73) Assignee: Allergan Sales, LLC, Irvine, CA (US)

(21) Appl. No.: 17/765,862

(22) PCT Filed: Sep. 30, 2020
(86) PCT No.: PCT/US2020/053461

§ 371 Date: Apr. 1, 2022

Related U.S. Application Data
(60) Provisional application No. 62/909,049, filed on Oct. 1, 2019.

(51) Int. Cl. A61K 9/50; A61K 38/17; C08G 81/00; A61K 9/00

(52) U.S. Cl. CPC A61K 9/5031; A61K 38/1709; C08G 81/00; A61K 9/0051; C08G 2230/00

(57) ABSTRACT

Provided herein are poly(ether ester) multi-block copolymers (PEE-MBCP). Also provided herein are injectable delivery systems or pharmaceutical compositions, comprising a PEE-MBCP provided herein, either alone or in combination with a binding protein, such as abicipar. Also provided herein are methods of using these injectable delivery systems or pharmaceutical compositions provided herein for the treatment of ocular disorders.

### CROSS-REFERENCE TO RELATED APPLICATIONS

This application claims the benefit of U.S. Provisional Application No. 62/909,049 filed on Oct. 1, 2019, the disclosure of which is hereby incorporated by reference in its entirety.

### BRIEF DESCRIPTION OF THE SEQUENCE LISTING

Incorporated herein by reference in its entirety is a Sequence Listing entitled, “21347-WO-PCT-OCU_ST25”, comprising 1 sequence. The Sequence listing has been submitted herewith in ASCII text format via EFS. The Sequence Listing was first created on Sep. 26, 2020 and is 4,096 bytes in size.

### BACKGROUND OF THE INVENTION

Proteins such as ranibizumab, bevacizumab and aflibercept have been successful in treating ocular disease. Unfortunately, water soluble protein drugs have very poor bioavailability from topical or systemic administration due to poor permeability, the blood-retinal barriers, their large molecular weight and systemic degradation. This requires that they be administered by direct local drug administration such as intravitreal or periocular injection. Unfortunately, relative to the duration of therapy, proteins have a relatively short half-life in the vitreous. This requires multiple intravitreal injection for treatment. However, multiple intraocular injections of this sort may lead to poor patient compliance and also increases the risk of intravitreal hemorrhages, retinal and vitreous detachment, and endophthalmitis. Additionally, the high peak concentrations resulting from this type of pulsed dosing may lead to toxicities. For these reasons there is a very significant unmet medical need for sustained intraocular macromolecule delivery systems.

### SUMMARY OF THE INVENTION

Provided herein are pharmaceutical compositions for the treatment of an ocular disorder in a patient in need thereof, comprising (a) a biologically active compound; and (b) a biodegradable, semi-crystalline, phase separated, thermoplastic poly(ether ester) multi-block copolymer;

wherein said biologically active compound is abicipar pegol,

wherein said multi-block copolymer comprises (i) an amorphous hydrolysable pre-polymer (A) segment having the following formula: (R¹R²ₙR³)_{q}; and (ii) a semi crystalline hydrolysable pre polymer (B) segment having the following formula: (R⁴ₚR⁵R⁶ₚ); arranged according to Formula (PEE-MBCP):

[(R¹R²ₙR³)_{q}]ᵣ[(R⁴ₚR⁵R⁶ₚ)]s (Formula PEE-MBCP)

wherein each segment is linked by a 1,4 butanediisocyanate chain extender,

wherein said segments are randomly distributed over the polymer chain;

wherein
R¹ and R³ are each

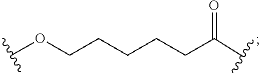 [SMILES: *OCCCCCC(*)=O]

R² is

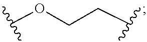 [SMILES: *CCO*]

R⁴ and R⁶ are each

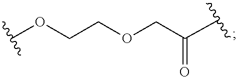 [SMILES: *OCCOCC(*)=O]

R⁵ is

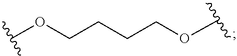

wherein
n, being the number of repeating R² moieties, is about 22 to about 23;
p, being the number of repeating R⁴ and R⁶ moieties, is about 11.5;
q, being the molecular weight of the (R¹R²ₙR³) block, is about 2000 g/mol;
r, being the weight fraction of pre-polymer (A) segment relative to the total amount of pre-polymer (A) and (B), is about 60%; and
s, being the weight fraction of pre-polymer (B) segment relative to the total amount of pre-polymer (A) and (B), is about 40%;

wherein said biologically active compound is encapsulated in a matrix comprising said multi-block copolymer; wherein said multi-block copolymer has a T_{g} of 37° C. or less and a Tₘ of 50-110° C. under physiological conditions, and wherein said multi-block copolymer has an intrinsic viscosity of about 0.8 dl/g.

A pharmaceutical composition for the treatment of an ocular disorder in a patient in need thereof, comprising (a) a biologically active compound; and (b) a biodegradable, semi-crystalline, phase separated, thermoplastic poly(ether ester) multi-block copolymer;

wherein said biologically active compound is abicipar pegol,

wherein said multi-block copolymer comprises (i) an amorphous hydrolysable pre-polymer (A) segment having the following formula: (R¹R²ₙR³)_{q}; and (ii) a semi crystalline hydrolysable pre polymer (B) segment having the following formula: (R⁴ₚR⁵R⁶ₚ); arranged according to Formula (PEE-MBCP):

[(R¹R²ₙR³)_{q}]r[(R⁴ₚR⁵R⁶ₚ)]s (Formula PEE-MBCP)
wherein each segment is linked by a 1,4 butanediisocyanate chain extender,
wherein said segments are randomly distributed over the polymer chain;

wherein

R¹ and R³ are each

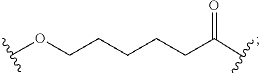 [SMILES: *OCCCCCC(*)=O]

R² is

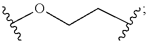 [SMILES: *CCO*]

R⁴ and R⁶ are each

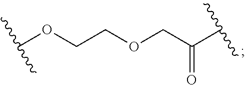 [SMILES: *OCCOCC(*)=O]

R⁵ is

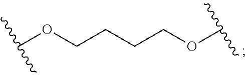

wherein
n, being the number of repeating R² moieties, is about 20 to about 25;
p, being the number of repeating R⁴ and R⁶ moieties, is about 10 to about 13.5;
q, being the molecular weight of the (R¹R²ₙR³) block, is about 1800 to about 2200 g/mol; and
the ratio r/s is 1.1-2.0, wherein r is the weight fraction of pre-polymer (A) segment and s is the weight fraction of pre-polymer (B) segment, relative to the total amount of pre polymer (A) and (B); and

wherein said biologically active compound is encapsulated in a matrix comprising said multi-block copolymer; wherein said multi-block copolymer has a T_{g} of 37° C. or less and a Tₘ of 50-110° C. under physiological conditions.

Also provided herein are pharmaceutical compositions in the form of a plurality of polymeric microspheres that are each not less than about 20 gm in diameter.

Also provided herein are pharmaceutical compositions in the form of a plurality of polymeric microspheres that are at least 20 μm in diameter.

Also provided herein are pharmaceutical compositions in the form of a plurality of polymeric microspheres which comprise about 4% to about 6% w/w of a biologically active compound.

Also provided herein are pharmaceutical compositions in the form of a plurality of polymeric microspheres which comprise about 4% w/w of a biologically active compound.

Also provided herein are pharmaceutical compositions in the form of a plurality of polymeric microspheres which comprise about 5% w/w of a biologically active compound.

Also provided herein are pharmaceutical compositions in the form of a plurality of polymeric microspheres which comprise about 6% w/w of a biologically active compound.

A biodegradable, semi-crystalline, phase separated, thermoplastic poly(ether ester) multi-block copolymer comprising (i) an amorphous hydrolysable pre-polymer (A) segment having the following formula: (R¹R²ₙR³)_{q}; and (ii) a semi crystalline hydrolysable pre polymer (B) segment having the following formula: (R⁴ₚR⁵R⁶ₚ); arranged according to Formula (PEE-MBCP):

[(R¹R²ₙR³)_{q}]r[(R⁴ₚR⁵R⁶ₚ)]s (Formula PEE-MBCP)

wherein each segment is linked by a 1,4 butanediisocyanate chain extender,

wherein said segments are randomly distributed over the polymer chin;

wherein
R¹ and R³ are each

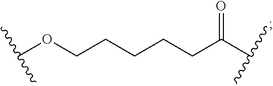 [SMILES: *OCCCCCC(*)=O]

R² is

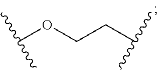 [SMILES: *CCO*]

R⁴ and R⁶ are each

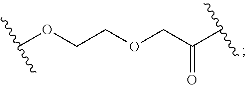 [SMILES: *OCCOCC(*)=O]

R⁵ is

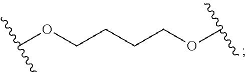

wherein
n, being the number of repeating R² moieties, is about 22 to about 23;
p, being the number of repeating R⁴ and R⁶ moieties, is about 11.5;
q, being the molecular weight of the (R¹R²ₙR³) block, is about 2000 g/mol;
r, being the weight fraction of pre-polymer (A) segment relative to the total amount of pre-polymer (A) and (B), is about 60%; and
s, being the weight fraction of pre-polymer (B) segment relative to the total amount of pre-polymer (A) and (B), is about 40%;

wherein said multi-block copolymer has a T_{g} of 37° C. or less and a Tₘ of 50-110° C. under physiological conditions, and wherein said multi-block copolymer has an intrinsic viscosity of about 0.8 dl/g.

Also provided herein are injectable delivery systems, wherein the injectable delivery system comprises a PEE-MBCP as described herein.

Also provided herein are methods of treating an ocular disease, comprising administering to a subject in need thereof a pharmaceutical composition or an injectable delivery system provided herein.

Also provided herein are methods of improving visual performance of an eye, comprising administering to a subject in need thereof an injectable pharmaceutical composition or delivery system provided herein.

Also provided herein are methods of extending the duration of efficacious release of a therapeutic agent, comprising administering a pharmaceutical composition or an injectable delivery system provided herein by intraocular injection whereby the therapeutic agent is slowly released from the delivery system at a rate leading to therapeutically effective concentrations of the therapeutic agent in the vitreous.

Also provided herein are methods of inhibiting retinal leakage and/or edema by administering a pharmaceutical composition or delivery system comprising a biodegradable polymer matrix and a binding protein via intraocular injection whereby the therapeutic agent is slowly released from the delivery system at a rate leading to therapeutically effective concentrations of the therapeutic agent within the vitreous.

Also provided herein are methods of reducing inflammation of an eye segment caused by intraocular injection to an eye, comprising administering a pharmaceutical composition or an injectable delivery system provided herein by intraocular injection whereby inflammation of the eye segment caused by intraocular injection is reduced.

Also provided herein are methods of decreasing aggregation of a therapeutic agent in a pharmaceutical composition or an injectable delivery system described herein, comprising preparing a pharmaceutical composition or an injectable delivery system provided herein whereby aggregation of the therapeutic agent in the said composition or said injectable delivery system is decreased.

Also provided herein are any of the aforementioned methods in which the pharmaceutical composition, injectable delivery system and/or binding protein comprises abicipar.

### BRIEF DESCRIPTION OF THE FIGURES

FIG. 1 Generic compositions of hydrophilic phase separated segmented multi-block copolymers composed of a crystalline poly(L-lactide) block in combination with an amorphous poly(ϵ-caprolactone)-PEG-poly(ϵ-caprolactone) block, e.g. PCL05.

FIG. 2 In vitro release profiles of three batches of abicipar pegol from PCL05 microspheres loaded with approximately 5% abicipar pegol.

FIG. 3 Vitreous humor levels achieved from an intravitreal injection of 10 mg of PCL05 abicipar pegol microspheres in rabbits and monkeys over 4 months (PK14056 and TX14051).

FIG. 4 Retinal adhesions and retinal detachments observed with PCL05 abicipar pegol microspheres when dosed in monkeys (10 mg of PCL05 containing 520 μg of abicipar pegol in a 50 μl injection was administered by intravitreal injection) (TX14051).

FIG. 5 Epiretinal membranes observed in the NZR rabbit vitreous upon administration of PLCO5 abicipar pegol microspheres (10 mg of PCL05 containing 520 μg of abicipar pegol in a 50 μl injection was administered by intravitreal injection) (TX13123).

FIG. 6 In vitro erosion of PCL05 (50CP10C20-LL40): experimental data up to 12 months and extrapolation of the experimental data up to complete erosion.

FIG. 7 In vitro erosion of microspheres composed of various L-MBCP, I-MBCP and SC-MBCP polymers. PCL05 (50CP10C20-LL40) is included as reference.

FIG. 8 In vitro erosion of microspheres composed of D-MBCP polymers containing various poly(e-caprolactone)-PEG-poly(ϵ-caprolactone) based counter blocks. PCL05 (50CP10C20-LL40) is included as reference.

FIG. 9 Generic compositions of hydrophilic phase separated segmented multi-block copolymers composed of a crystalline polydioxanone block in combination with an amorphous poly(e-caprolactone)-PEG-poly(e-caprolactone) block, e.g. PCD21.

FIG. 10 In vitro release profiles of abicipar pegol from PCD21 microspheres loaded with 6% abicipar pegol (MS16-081).

FIG. 11 Vitreous cell score of the rabbit vitreous after administration of 10 mg PCD21 polymer-only microspheres into the rabbit vitreous out to day 225 (TX15024).

FIG. 12 Appearance of polymer-only microspheres composed of PCD21 (Group 4) and 30LCP10LC20-L/LL40 (Group 7) at 4 and 8 months (TX15024).

FIGS. 13A-B—Shows sustained suppression of retinal leak by abicipar pegol

PCD21 microspheres (Plate A—fluorescein angiograms; and Plate B—retinal leak areas) in a rabbit model of persistent retinal vascular leak).

FIG. 14 Erosion of PCD21 microspheres loaded with abicipar pegol in the eye (eye #11199) in a rabbit persistent retinal vascular leak model.

FIGS. 15A-B—Suppression of retinal leak in the eye treated with abicipar pegol loaded PCD21 microspheres in a Dutch Belted rabbit persistent retinal vascular leak model.

FIG. 16 Fundus images of abicipar pegol loaded PCD21 microspheres in the eye in a Dutch Belted rabbit persistent retinal vascular leak model.

FIG. 17 Vitreous cell grades in TX16015, a single dose intravitreal toxicity study in primates.

FIG. 18 Sustained delivery of abicipar pegol with PCD21 microspheres to primate ocular tissues (PK16031).

FIGS. 19A-C—DSC thermograms of RCP-15126 (Plate A), RCP-15125 (Plate B) and (RCP-1524 (Plate C) multi-block copolymers.

FIG. 20 SEM images of the different abicipar pegol loaded microsphere batches, prepared with poly(p-dioxanone) based multi-block copolymers with different poly(p-dioxanone) Mₙs and contents (i.e. block ratios).

FIG. 21 Abicipar pegol in vitro release (IVR) from microsphere based on poly(p-dioxanone) based multi-block copolymers with different poly(p-dioxanone) Mn's and block ratios.

FIG. 22 SEM images of the different polymer-only microsphere batches, prepared with poly(p-dioxanone) based multi-block copolymers with different composition of the hydrophilic block (PEG Mₙ, PEG content, poly(ϵ-caprolactone) chain length) and block ratio).

FIG. 23 In vitro erosion kinetics of polymer-only microspheres composed of 57CP10C20-D28, 35CP15C20-D24, 50CP15C20-D24, 20CP30C40-D23 (50CP10C20-LL40 was used as reference).

FIG. 24 Effect of molecular weight of poly(e-caprolactone) chains on in vitro erosion of several poly(p-dioxanone) based multi-block copolymers (50CP10C20-LL40 was used as reference).

FIG. 25 SEM photographs of polymer-only microspheres prepared of 60CP10C20-Dxx multi-block copolymers composed of polydioxanone-blocks with different molecular weight (Mₙ).

FIG. 26 Effect of molecular weight (Me) of the polydioxanone pre-polymer block on the melting enthalpy of 60CP10C20-Dxx multi-block copolymers and polymer-only microspheres composed thereof.

FIGS. 27A-B—Effect of molecular weight Mₙ of the polydioxanone block of 60CP10C20-Dxx multi-block copolymers on abicipar pegol release kinetics (A) shows cumulative in vitro release of abicipar pegol from abicipar pegol microspheres and (B) shows the burst release of abicipar pegol microspheres.

FIGS. 28A-B—SEM images of polymer-only microspheres prepared of 60CP10C20-Dxx polymers containing polydioxanone blocks with Mₙ 2116 g/mol (RCP-1710), 2356 g/mol (RCP-1718) and 2806 g/mol (RCP-1714) (panel A) and their in vitro erosion kinetics (50CP10C20-LL40 is included as a reference) (panel B).

FIGS. 29A-B - SEM images of abicipar pegol loaded microspheres prepared of 60CP10C20-Dxx polymers containing polydioxanone blocks with Mₙ 2538 g/mol (RCP-1728B, 2887 g/mol (RCP-1812) and 3840 g/mol (RCP1807) (Plate A) and their in vitro abicipar pegol release kinetics (Plate B).

FIG. 30 Cumulative in vitro release of abicipar pegol from PCD21-based abicipar pegol microspheres manufactured at a scale of 2.5 g.

FIGS. 31A-B—Cumulative in vitro release of abicipar pegol from PCD21-based abicipar pegol microspheres manufactured at a scale of 25 g. Plate A represents total abicipar pegol released; Plate B represents intact abicipar pegol released (Batch nr. 060A-180612-04).

FIGS. 32A-C—Cumulative in vitro release of intact abicipar pegol from three batches of PCD21-based abicipar pegol microspheres manufactured at a scale of 25 g (batch nrs. 060A-181105-05 (RCP-1815—Plate A); 060A-181119-05 (RCP-1816—Plate B); 060A-181123-05) (RCP-1816—Plate C).

### DEFINITIONS

Listed below are definitions of various terms used to describe this invention. These definitions apply to the terms as they are used throughout this specification and claims, unless otherwise limited in specific instances, either individually or as part of a larger group.

The term “about” generally indicates a possible variation of no more than 10%, 5%, or 1% of a specified value. For example, “about 25 mg/kg” will generally indicate, in its broadest sense, a value of about 22.5-27.5 mg/kg, i.e., 25 ±2.5 mg/kg.

The term “biodegradable” as used herein refers to a material that will break down actively or passively over time by simple chemical processes, by action of body enzymes or by other similar biological activity mechanisms. The term “biodegradable polymer” as used herein refers to a polymer or polymers which degrade in vivo as a result of the breaking of chemical bonds within the polymer (i.e., chemical chain scission), resulting in a reduction in the molecular weight of the polymer(s), which occurs over time concurrently with or subsequent to release of the therapeutic agent. A biodegradable polymer may be a homopolymer, a copolymer, or a polymer comprising more than two different polymeric units.

The term “bioerodible” as used herein refers to chemical or enzymatic solubilization of a material in vivo with or without changes in the chemical structure of the material. The term “bioerodible polymeric matrix” refers to a polymeric matrix that undergoes mass loss in vivo, with or without reduction of the molecular weight of the polymer(s) contained in the matrix, and wherein the mass loss (erosion) over time occurs concurrently with or subsequent to release of the therapeutic agent.

Coefficient of variation (CV) refers to standard deviation, which is expressed in % of the mean.

The term “designed ankyrin repeat protein” or “DARPin®” as used herein refers to a class of small protein therapeutic agents derived from natural ankyrin repeat proteins. One preferred example of a DARPin® is abicipar.

The term “engineered lipocalin” or “Anticalin®” as used herein refers to an artificial protein derived from human lipocalins. Anticalins are structurally characterized as barrels formed by eight antiparallel n-strands pairwise connected by loops and an attached α-helix. p The term “particle” as used herein refers to an extremely small constituent (e.g., nanoparticle, microparticle, or in some instances larger) that may contain in whole or in part at least one therapeutic agent. A particle may contain therapeutic agent(s) in a core surrounded by a coating. Therapeutic agent(s) also may be dispersed throughout the particle. Therapeutic agent(s) also may be adsorbed into the particle. A particle may be of any order release kinetics, including zero order release, first order release, second order release, delayed release, sustained release, immediate release, etc., and any combination thereof. A particle may include, in addition to a therapeutic agent(s), any of those materials routinely used in the art of pharmacy and medicine, including, but not limited to, erodible material, non-erodible material, biodegradable material, non-biodegradable material or a combination thereof. A particle may be of virtually any shape.

The term “peptide” as used herein refers to a molecule of two or more amino acids or amino acid analogs linked by a chemical bond formed when the carboxyl group (—COOH) of one amino acid reacts with the amino group (—NH₂) of another amino acid, forming the sequence CONH and releasing a molecule of water (H₂O) (i.e., peptide bond).

The term “plurality” as used herein means more than one.

The term “polypeptide” is used herein in its broadest sense to refer to a sequence of subunit (i.e., one or more chains of two or more) amino acids, amino acid analogs or peptidomimetics, wherein the subunits are linked by peptide bonds.

The term “protein” as used herein refers to a large complex molecule or polypeptide composed of amino acids, wherein at least part of the polypeptide has, or is able to acquire a defined three-dimensional arrangement by forming secondary, tertiary, or quaternary structures within and/or between its polypeptide chain(s). If a protein comprises two or more polypeptides, the individual polypeptide chains may be linked non-covalently or covalently, e.g. by a disulfide bond between two polypeptides. A part of a protein, which individually has, or is able to acquire a defined three-dimensional arrangement by forming secondary or tertiary structures, is termed a “protein domain.”

As is known in the art, “abicipar” (also known as MP-0112 and AGN-150998) is a VEGF-A specific, “designed ankyrin repeat protein”, or “DARPin®”, being developed as an ocular anti-neovascularization agent, and is in Ph 3 clinical trials for the treatment of ocular disorders including age-related macular degeneration and diabetic macular edema, among other ocular indications. Abicipar has a CAS Registry Number of 1327278-94-3; an empirical formula of C₆₂₈H₉₈₅N₁₇₅O₂₀₃S_{2 [}C₂H₄O]n; and has an approximate molecular weight of 34 kDa. Abicipar comprises SEQ ID NO:1. One example of a pharmaceutical composition comprising abicipar is abicipar pegol for injection. Abicipar pegol comprises SEQ ID NO:1 which is conjugated to a maleimide-coupled polyethylene glycol (α-[3-(3-maleimido-1-oxopropyl)amino]propyl-ω-methoxy-polyoxyethylene) at its C-terminus via a peptide bond to a polypeptide linker and a C-terminal Cys residue, wherein the polyethylene glycol has a molecular weight of about 20 kDa, and which further has an N-terminal capping module comprising an Asp residue at position 5.

While therapeutic agents may be referred to herein in their neutral forms, in some embodiments, these compounds are used in a pharmaceutically acceptable salt form. As used herein, “pharmaceutically acceptable salts” refers to derivatives of the disclosed compounds wherein the parent compound is modified by converting an existing acid or base moiety to its salt form. Lists of suitable salts are found in Remington's Pharmaceutical Sciences, 17th ed., Mack Publishing Company, Easton, Pa., 1985, p. 1418 and Journal of Pharmaceutical Science 1977, 66(1), 1-19, each of which is incorporated herein by reference in its entirety.

### DETAILED DESCRIPTION OF THE INVENTION

Developing protein and peptide delivery systems remains a highly challenging area. These compounds often require intact quaternary structures for their biologic activity. Maintaining the compound's structural integrity within the formulation, during the manufacturing processes, and throughout the performance of the sustained delivery system is quite complex. Proteins can be denatured by heat, shear forces, pH extremes, organic solvents, hydrophobic interfaces, freezing and drying. Proteins or peptides can also be susceptible to damage from irradiation utilized in the terminal sterilization of the final drug product. Proteins or peptides may interact with many of the hydrophobic polymers used in the fabrication of sustained delivery systems, becoming adsorbed, degraded, aggregated or denatured. This may lead to loss of activity and immunogenicity. Extensive efforts have been made to achieve sustained release protein formulations, but very little success has been obtained in delivering active proteins for a period of more than two months.

Intravitreal sustained delivery of proteins is particularly difficult and there are five main requirements that have been technically challenging. To date very few systems meet any of these requirements and none meet all.

These are: (i) Prevention of protein aggregation and preservation of protein activity during formulation and manufacturing; (ii) Sustained release of intact monomeric protein for at least four months into the vitreous with minimal burst; (iii) Protein stability in the drug delivery system—chemical and physical (aggregation) stability are key issues that need to be overcome.

Aggregation is a key risk for immunogenicity an inflammation; (iv) Acute and chronic tolerability of the system in the sensitive posterior segment of the eye. Many protein delivery systems suffer from acute or chronic inflammation, epiretinal membrane formation, cataract or migration to the anterior chamber; and (v) Timely bioerosion of erodible delivery systems. The accumulation of residual polymer should be controlled to mitigate any tolerability risk following repeated administration of the formulation.

The inventors have described, for the first time, an intraocular delivery system or pharmaceutical composition capable of delivering a sustained, and continuous, release of a biologically active compound, in this instance the therapeutic anti-neovascular protein composition, abicipar pegol, over the course of at least four to eight months, that is capable of achieving a high-drug load of about 5 to 6%, with high encapsulation efficiencies, and with minimal levels of pre-mature erosion, pre-mature burst release, aggregation, and accumulation. In addition, the intraocular delivery systems or pharmaceutical compositions described herein surprisingly resulted in significant improvements in the tolerability of abicipar pegol as evidenced by the absence of observed intraocular inflammation or adverse events post-injection beyond the basal level observed for non-abicipar pegol loaded compositions over a follow-up period of at least 16 months.

A pharmaceutical composition is a formulation that contains at least one active pharmaceutical ingredient, as well as, for example, one or more polymers, excipients, buffers, carriers, stabilizers, preservatives, or bulking agents, and is suitable for administration to a subject in order to achieve a desired diagnostic result or therapeutic effect.

Intraocular injection of particle suspensions, polymeric particles, or polymeric depots, which contain an active pharmaceutical ingredient or simply a placebo, may elicit serious adverse events (SAEs). These SAEs may manifest as inflammation, a severe immune response, lens opacities, retinal separation, macrophage incursion, clouding of the vitreous, cells in the vitreous, particles moving anteriorly to potentially cause other SAEs, or a combination thereof. Likewise, intracameral injection of particle suspensions may elicit SAEs.

### Multi-Block Co-Polymers

Disclosed herein is a biodegradable, phase separated, thermoplastic poly(ether ester) multi-block copolymers

[(R¹R²ₙR³)_{q}]ᵣ[(R⁴ₚR⁵R⁶ₚ)](PEE-MBCP) comprising or consisting of segments linked by a 1,4-butanediisocyanate chain extender, said segments being selected from the group consisting of an amorphous hydrolysable (R¹R²ₙR³)_{q} pre-polymer (A) segment and a semi-crystalline hydrolysable (R⁴ₚR⁵R⁶ₚ) pre-polymer (B) segment with the proviso that said multi-block copolymer comprises at least one pre-polymer (A) segment and at least one pre-polymer (B) segment, wherein

said PEE-MBCP under physiological conditions has a T_{g} of 37° C. or less and a Tₘ of 50-110° C.; and

the segments are randomly distributed over the polymer chain; and wherein

R¹ and R³ are each

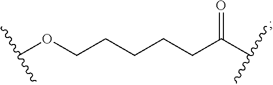 [SMILES: *OCCCCCC(*)=O]

R² is

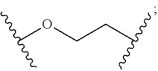 [SMILES: *CCO*]

R⁴ and R⁶ are each

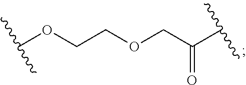 [SMILES: *OCCOCC(*)=O]

R⁵ is

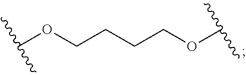 [SMILES: *OCCCCO*]

“n”, being the number of repeating R² moieties, is about 20 to about 120;

“p”, being the number or repeating R⁴ and R⁶ moieties, is about 10 or more;

“q”, being the molecular weight of the (R¹R²ₙR³) block is about 1500 to about 9000 g/mol; and

the ratio r/ s is about 0.1 to about 2.5, wherein “r” is the weight fraction of pre-polymer (A) segment and “s” is the weight fraction of pre-polymer (B) segment.

As disclosed herein, “n” is about 20 to about 70, about 20 to about 46, about 20 to about 38, about 21 to about 28, or a range bounded by any two of these values. In some embodiments, “n” is about 22. In some embodiments, “n” is about 23.

As disclosed herein, “p” is about 10 to about 25, about 10 to about 20, about 10 to about 15, about 10 to about 13, or a range bounded by any two of these values. In some embodiments, “p” is about 10.5. In some embodiments, “p” is about 11.5. In some embodiments, “p” is about 12.5.

As disclosed herein, “q” is about 1500 to about 7000, about 1500 to about 6500, about 1550 to about 6000, about 1550 to about 5500, about 1550 to about 5000, about 1600 to about 4500, about 1600 to about 4000, or a range bounded by any two of these values.

As disclosed herein, “q” is about 1800 to about 2150, about 1850 to about 2150, about 1850 to about 2100, about 1900 to about 2100, about 1900 to about 2050, about 1950 to about 2050, about 1950 to about 2000, or a range bounded by any two of these values. In some embodiments, “q” is about 2000.

As disclosed herein, “r” is about 20 to about 70, about 40 to about 60, about 45 to about 70, or a range bounded by any two of these values.

100461 As disclosed herein, “r” is about 55 to about 66, about 55 to about 65, about 56 to about 65, about 56 to about 64, about 57 to about 64, about 57 to about 63, about 58 to about 63, about 58 to about 62, about 59 to about 62, about 59 to about 61, or a range bounded by any two of these values. In some embodiments, “r” is about 60.

As disclosed herein, “s” is about 30 to about 80, about 35 to about 70, about 40 to about 60, or a range bounded by any two of these values.

As disclosed herein, “s” is about 37 to about 44, about 37 to about 43, about 38 to about 43, about 38 to about 42, about 39 to about 42, about 39 to about 41, or a range bounded by any two of these values. In some embodiments, “s” is about 40.

As disclosed herein, “n” is about 22 to about 24; “p” is about 10.5; “q” is about 2000; “r” is about 60; and “s” is about 40.

As disclosed herein, “n” is about 22 to about 23; “p” is about 11.5; q is about 2000; “r” is about 60; and “s” is about 40.

As disclosed herein, “n” is about 22 to about 23; “p” is about 12.5; q is about 2000; “r” is about 60; and s is about 40.

As disclosed herein, the PEE-MBCP in which “n” is about 22 to about 23; “p” is about 11.5; “q” is about 2000 g/mol; “r” is about 60%; and “s” is about 40%;, is referred to as PCD21.

As disclosed herein, the PEE-MBCP comprises a random polymeric mixture of block [(R¹R²ₙR³)_{q}] with block (R⁴ₚR⁵R⁶ₚ) to form a PEE-MBCP of formula [(R¹R²ₙR³)_{q}], [(R⁴ₚR⁵R⁶ₚ)]₈, wherein

(R¹R²ₙR³) is

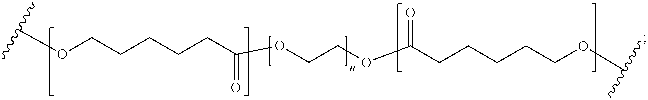

R⁴ and R⁶ are each;

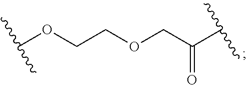 [SMILES: *OCCOCC(*)=O]

R⁵ is

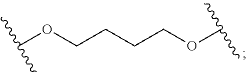 [SMILES: *OCCCCO*]

“n”, being the number of repeating R² moieties, is about 22 to about 23, such as about 22 or about 23;

“p”, being the number or repeating R⁴ and R⁶ moieties, is about 10.5 to about 12.5, such as about 10.5, about 11.5 or about 12.5;

“q”, being the molecular weight of the (R¹R²ₙR³) block is about 2000 g/mol;

“r” is about 60%; and

“s” is about 40%.

### Injectable Delivery Systems

In one aspect, provided herein are injectable delivery systems comprising a PEE-MBCP provided herein.

In some embodiments, the PEE-MBCP is in the form of an implant. The form of the implant includes, but is not limited to a rod, a film, a sheet, a disc, a gel, a solution, a particle, a PEE-MBCP depot, or a combination thereof. Non-limiting examples of particles include spheres. Spheres include, but are not limited to, microspheres and nanospheres.

In some embodiments, the PEE-MBCP is in the form of a plurality of polymeric microspheres that are each not less than about 20 μm in diameter, wherein the polymeric microspheres comprise the PEE-MBCP. In some embodiments, the injectable delivery systems comprise a therapeutic agent. In some embodiments, the injectable delivery systems comprise one therapeutic agent. In some embodiments, the injectable delivery systems comprise at least one therapeutic agent. In some embodiments, the injectable delivery systems comprise more than one therapeutic agent. Such therapeutic agents include, but are not limited to, small chemical drugs and biologic agents. Small chemical drugs include, but are not limited to, a synthetic derivative of cremastranone (SH-11037) and an oligonucleotide. Non-limiting examples of oligonucleotides include deoxyribonucleic acid (DNA) and ribonucleic acid (RNA). RNA includes, but is not limited to, antisense RNA (asRNA), small interfering RNA (siRNA) and microRNA (miRNA). Biologic agents include, but are not limited to, a peptide and a protein. Proteins include, but are not limited to, an antibody and an antibody mimetic protein.

Non-limiting examples of antibodies include bevacizumab (Avastin®) and ranibizumab (Lucentis®). Antibody mimetic proteins include, but are not limited to, pegaptanib (Macugen®), aflibercept (Eylea®), designed ankyrin repeat proteins (DARPin®) and engineered lipocalins (Anticalin®). DARPin® proteins include, but are not limited to, abicipar and abicipar conjugated to polyethylene glycol (abicipar pegol). Anticalin® proteins include, but are not limited to, PRS-050 and PRS-055.

In some embodiments, the plurality of polymeric microspheres comprises a therapeutic agent. In some embodiments, the plurality of polymeric microspheres comprises about 1% to about 15% w/w of a therapeutic agent. In some embodiments, the plurality of polymeric microspheres comprises about 1% to about 5% w/w, about 5% to about 10% w/w, about 10% to about 15% w/w, about 1% to about 3% w/w, about 3% to about 5% w/w, about 5% to about 7% w/w, about 7% to about 9% w/w, about 9% to about 11% w/w, about 11% to about 13% w/w, or about 13% to about 15% of a therapeutic agent. In some embodiments, the plurality of polymeric microspheres comprises about 4% to about 7% w/w of a therapeutic agent. In some embodiments, the plurality of polymeric microspheres comprises about 4% w/w of a therapeutic agent. In some embodiments, the plurality of polymeric microspheres comprises about 5% w/w of a therapeutic agent. In some embodiments, the plurality of polymeric microspheres comprises about 6% w/w of a therapeutic agent. In some embodiments, the plurality of polymeric microspheres comprises about 7% w/w of a therapeutic agent.

In some embodiments, the plurality of polymeric microspheres comprises abicipar pegol. In some embodiments, the plurality of polymeric microspheres comprises about 1% to about 15% w/w of abicipar pegol. In some embodiments, the plurality of polymeric microspheres comprises about 1% to about 5% w/w, about 5% to about 10% w/w, about 10% to about 15% w/w, about 1% to about 3% w/w, about 3% to about 5% w/w, about 5% to about 7% w/w, about 7% to about 9% w/w, about 9% to about 11% w/w, about 11% to about 13% w/w, or about 13% to about 15%, of abicipar pegol. In some embodiments, the plurality of polymeric microspheres comprises about 4% to about 7% w/w abicipar pegol. In some embodiments, the plurality of polymeric microspheres comprises about 4% w/w abicipar pegol. In some embodiments, the plurality of polymeric microspheres comprises about 5% w/w abicipar pegol. In some embodiments, the plurality of polymeric microspheres comprises about 6% w/w abicipar pegol. In some embodiments, the plurality of polymeric microspheres comprises about 7% w/w abicipar pegol.

In some embodiments, the injectable delivery systems further comprise a pharmaceutically acceptable excipient.

In some embodiments, provided herein is an injectable delivery system comprising PCD21.

In some embodiments, provided herein is an injectable delivery system comprising PCD21 and abicipar pegol.

In some embodiments, provided herein is an injectable delivery system comprising 4-7% w/w abicipar pegol, and a PEE-MBCP comprising a random polymeric mixture of block [(R¹R²ₙR³)_{q}] with block (R⁴ₚR⁵R⁶ₚ) to form a PEE-MBCP of formula [(R¹R²nR³)_{q}]ᵣ[(R⁴ₚR⁵R⁶ₚ)]ₛ, wherein

(R¹R²ₙR³) is

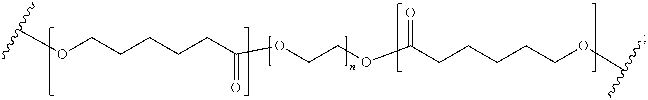

R⁴ and R⁶ are each

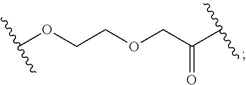 [SMILES: *OCCOCC(*)=O]

R⁵ is

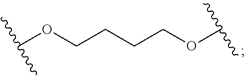 [SMILES: *OCCCCO*]

“n”, being the number of repeating R² moieties, is about 20 to about 120;

“p”, being the number or repeating R⁴ and R⁶ moieties, is about 10 or more;

“q”, being the molecular weight of the (R¹R²ₙR³) block is about 1500 to about 9000 g/mol; and

the ratio r/s is about 0.1 to about 2.5, wherein “r” is the weight fraction of pre-polymer (A) segment and s is the weight fraction of pre-polymer (B) segment, relative to the total amount of pre-polymer (A) and (B).

In some embodiments, provided herein is an injectable delivery system comprising 4-7% w/w abicipar pegol, and a PEE-MBCP comprising a random polymeric mixture of block [(R¹R²ₙR³)_{q}] with block (R⁴ₚR⁵R⁶ₚ) to form a PEE-MBCP of formula [(R¹R²_{nR}³)_{q}]ᵣ[(R⁴ₚR⁵R⁶ₚ)]ₛ, wherein

(R¹R²ₙR³) is

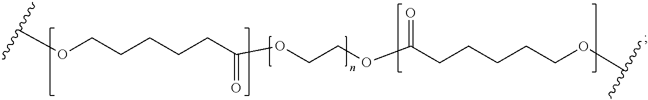

R⁴ and R⁶ are each

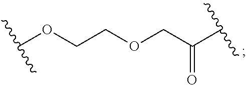 [SMILES: *OCCOCC(*)=O]

R⁵ is

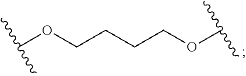 [SMILES: *OCCCCO*]

“n”, being the number of repeating R² moieties, is about 22 to about 23, such as about 22 or about 23;

“p”, being the number or repeating R⁴ and R⁶ moieties, is about 10.5 to about 12.5, such as about 10.5, about 11.5, or about 12.5;

“q”, being the molecular weight of the (R¹R²ₙR³) block is about 2000 g/mol;

“r” is about 60%; and

“s” is about 40%.

### Methods

Provided herein are methods of treating an ocular disease, comprising administering to a subject in need thereof an injectable delivery system provided herein with a therapeutic agent.

Also provided herein are methods of improving visual performance of an eye, comprising administering to a subject in need thereof an injectable delivery system provided herein with a therapeutic agent.

In some embodiments, the injectable delivery system is administered by intraocular injection.

In some embodiments, the intraocular injection is intravitreal, subretinal, subconjunctival, or intracameral.

Also provided herein are methods of extending efficacy duration of a therapeutic agent, comprising administering an injectable delivery system provided herein by intraocular injection whereby the therapeutic agent is slowly released from the delivery system at a rate leading to therapeutically effective concentrations of the therapeutic agent within the vitreous.

Also provided herein are methods of extending the duration of efficacious release of a therapeutic agent comprising administering an injectable delivery system comprising a biodegradable intraocular composition comprising or consisting of a biodegradable polymer matrix and a binding protein associated within the biodegradable polymer matrix, wherein the implant provides continuous release of said binding protein in a biologically active form post injection within an eye of a mammal, preferably a primate, for at least about 90 days, at least about 100 days, at least about 110 days, at least about 120 days, at least about 130 days, at least about 140 days, at least about 150 days, at least about 160 days, at least about 170 days, at least about 180 days, at least about 190 days, at least about 200 days, at least about 210 days, at least about 220 days, at least about 230 days, at least about 240 days, at least about 250 days, at least about 260 days, at least about 270 days, at least about 280 days, at least about 290 days, at least about 300 days, at least about 310 days, at least about 320 days, at least about 330 days, at least about 340 days, at least about 350 days, at least about 360 days, at least about 13 months, at least about 14 months, at least about 15 months, or at least about 16 months in primates. In a preferred embodiment, the biodegradable polymer matrix is PCD21, and the binding protein is abicipar.

Also provided herein are methods of decreasing aggregation of a therapeutic agent in an injectable delivery system, comprising preparing an injectable delivery system or pharmaceutical composition provided herein with a therapeutic agent whereby aggregation of the therapeutic agent in the injectable delivery system is decreased. 100711 Also provided herein are methods of reducing inflammation of an eye segment caused by intraocular injection to an eye, comprising administering an injectable delivery system or pharmaceutical composition provided herein by intraocular injection whereby inflammation of the eye segment caused by intraocular injection is reduced.

In some embodiments, provided herein is a method of treating an ocular disease, comprising administering to a subject in need thereof an injectable delivery system or pharmaceutical composition comprising abicipar pegol, and PCD21.

In some embodiments, provided herein is a method of treating an ocular disease, comprising administering to a subject in need thereof an injectable delivery system comprising 4-7% w/w abicipar pegol, and a PEE-MBCP comprising a random polymeric mixture of block [(R¹R²ₙR³)_{q}] with block (R⁴ₚR⁵R⁶ₚ) to form a PEE-MBCP of formula [(R¹R²ₙR³)_{q}]ᵣ[(R⁴ₚR⁵R⁶ₚ)]ₛ, wherein

(R¹R²nR³) is

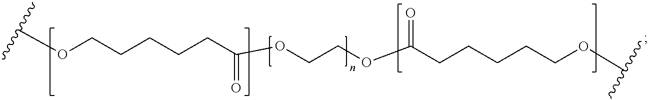

R⁴ and R⁶ are each

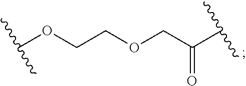 [SMILES: *OCCOCC(*)=O]

R⁵ is

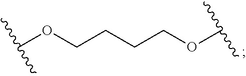 [SMILES: *OCCCCO*]

“n”, being the number of repeating R² moieties, is about 20 to about 120;

“p”, being the number or repeating R⁴ and R⁶ moieties, is about 10 or more;

“q”, being the molecular weight of the (R¹R²nR³) block is about 1500 to about 9000 g/mol; and

the ratio r/s is about 0.1 to about 2.5, wherein “r” is the weight fraction of pre-polymer (A) segment and s is the weight fraction of pre-polymer (B) segment, relative to the total amount of pre-polymer (A) and (B).

In some embodiments, provided herein is a method of treating an ocular disease, comprising administering to a subject in need thereof an injectable delivery system or pharmaceutical composition comprising 4-7% w/w abicipar pegol, and a PEE-MBCP comprising a random polymeric mixture of block [(R¹R²ₙR³)_{q}] with block (R⁴ₚR⁵R⁶ₚ) to form a PEE-MBCP of formula [(R¹R²ₙR³)_{q}]ᵣ,[(R⁴ₚR⁵R⁶ₚ)]ₛ, wherein

(R¹R²ₙR³) is

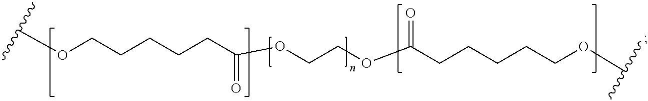

R⁴ and R⁶ are each

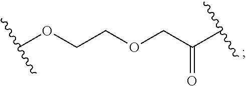 [SMILES: *OCCOCC(*)=O]

R⁵ is

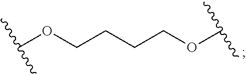 [SMILES: *OCCCCO*]

“n”, being the number of repeating R² moieties, is about 22 to about 23, such as about 22 or about 23;

“p”, being the number or repeating R⁴ and R⁶ moieties, is about 10.5 to about 12.5, such as about 10.5, about 11.5 or about 12.5;

“q”, being the molecular weight of the (R¹R²ₙR³) block is about 2000 g/mol;

“r” is about 60%; and

“s” is about 40%.

In some embodiments, the multi-block copolymers (MBCPs) described herein are unsolvated. In other embodiments, one or more of the

MBCPs are in solvated form. As known in the art, the solvate can be any of pharmaceutically acceptable solvent, such as water, ethanol, and the like.

Provided herein is a biodegradable, phase separated, thermoplastic poly(ether ester) multi-block copolymer [(R¹R²ₙR³)_{q}]ᵣ[(R⁴ₚR⁵R⁶ₚ)]ₛ (PEE-MBCP) comprising or consisting of segments linked by a 1,4-butanediisocyanate chain extender, said segments being selected from the group consisting of an amorphous hydrolysable (R¹R²ₙR³)_{q} pre-polymer (A) segment and a semi-crystalline hydrolysable (R⁴ₚR⁵R⁶ₚ) pre-polymer (B) segment with the proviso that said multi-block copolymer comprises at least one pre-polymer (A) segment and at least one pre-polymer (B) segment, wherein said PEE-MBCP under physiological conditions has a T_{g} of 37° C. or less and a Tₘ of 50-110° C.; and the segments are randomly distributed over the polymer chain; and wherein

R¹ and R³ are each

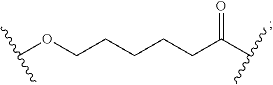 [SMILES: *OCCCCCC(*)=O]

R² is

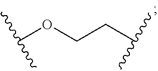 [SMILES: *CCO*]

R⁴ and R⁶ are each

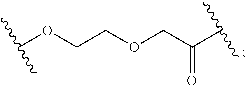 [SMILES: *OCCOCC(*)=O]

R⁵ is

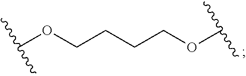 [SMILES: *OCCCCO*]

“n”, being the number of repeating R² moieties, is 20-120;

“p”, being the number of repeating R⁴ and R⁶ moieties is 10 or more;

“q”, being the molecular weight of the (R¹R²ₙR³) block is 1500-9000 g/mol; and

the ratio r/s is 0.1-2.5, wherein “r” is the weight fraction of pre-polymer (A) segment and “s” is the weight fraction of pre-polymer (B) segment, relative to the total amount of pre-polymer (A) and (B).

As disclosed herein, the PEE-MBCP herein has the following parameters, wherein “q” is 1500-7000 g/mol; “r” is 20-70%; and “s” is 30-80%.

As disclosed herein, the PEE-MBCP herein has an intrinsic viscosity of 0.7-0.9 dl/g.

As disclosed herein, the PEE-MBCP herein has an “n” of about 20 to about 70.

As disclosed herein, the PEE-MBCP herein has an “n” of about 21 to about 46.

As disclosed herein, the PEE-MBCP herein has a “p” of about 10 to about 15.

As disclosed herein, the PEE-MBCP herein has a “p” of about 10 to about 13.

As disclosed herein, the PEE-MBCP herein has a “q” of about 1550 to about 5000 g/mol.

As disclosed herein, the PEE-MBCP herein has a “q” of about 1600 to about 4000 g/mol.

As disclosed herein, the PEE-MBCP herein has an “r” of about 30 to about 65%.

As disclosed herein, the PEE-MBCP herein has an “r” of about 40 to about 60%.

As disclosed herein, the PEE-MBCP herein has an “s” of about 35 to about 70%.

As disclosed herein, the PEE-MBCP herein has an “s” of about 40 to about 60%.

As disclosed herein, the PEE-MBCP herein has an “n” of about 22 to about 23; has a “p” of about 10.5; has a “q” of about 2000 g/mol; has a “r” of about 60%; and has a “s” is about 40%.

As disclosed herein, the PEE-MBCP herein has an “n” of about 22 to about 23; has a “p” of about 11.5; has a “q” of about 2000 g/mol; has a “r” of about 60%; and has a “s” of about 40%.

As disclosed herein, the PEE-MBCP herein has an “n” of about 22 to about 23; has a “p” of about 12.5; has a “q” of about 2000 g/mol; has a “r” of about 60%; and has a “s” of about 40%.

As disclosed herein, provided herein is a composition for the delivery of at least one biologically active compound to a host, comprising at least one biologically active compound encapsulated in a matrix, wherein said matrix comprises at least one biodegradable, semi-crystalline, phase separated, thermoplastic PEE-MBCP multi-block copolymer as described herein.

As disclosed herein, the at least one biologically active compound is a non-peptide, non-protein, small sized drug, or a biologically active polypeptide.

As disclosed herein, the at least one biologically active compound is a recombinant binding protein comprising an ankyrin repeat domain.

As disclosed herein, the ankyrin repeat domain binds VEGF-Axxx with a Kd below 10⁹ M.

As disclosed herein, the binding protein comprises SEQ ID NO:1.

As disclosed herein, the binding protein further comprises a polyethylene glycol moiety having a molecular weight of at least 5 kDa.

As disclosed herein, the binding protein is conjugated at its C-terminus via a peptide bond to a polypeptide linker and a C-terminal Cys residue, wherein the thiol of the C-terminal Cys is further conjugated to a maleimide-coupled polyethylene glycol.

As disclosed herein, the maleimide-coupled polyethylene glycol is α-[3-(3-maleimido-1-oxopropyl)amino]propyl-ω-methoxy-polyoxyethylene.

As disclosed herein, the N-terminal capping module of the polypeptide of SEQ ID NO:1 comprises an Asp residue at position 5.

As disclosed herein, the injectable delivery system or composition comprising the PEE-MBCP as described herein has a PEE-MBCP in the form of a plurality of polymeric microspheres that are each not less than about 20 μm in diameter.

As disclosed herein, the plurality of polymeric microspheres comprise about 4% to about 6% w/w of the binding protein.

As disclosed herein, the plurality of polymeric microspheres comprise about 4% w/w, 5% w/w, or 6% w/w of the binding protein.

As disclosed herein, provided herein is a method of inhibiting binding between VEGF-Axxx and VEGFR-2 in a subject, comprising administering to an eye of a subject in need of such inhibition any one of the compositions or injectable delivery systems described herein.

As disclosed herein, provided herein is a method of treating a condition selected from age-related macular degeneration, neovascular age-related macular degeneration, diabetic macular edema, pathological myopia, branch retinal vein occlusion, or central retinal vein occlusion, the method comprising administering to an eye of a subject in need of such treatment any one of the compositions or injectable delivery systems described herein.

As disclosed herein, provided herein is a method of treating a condition selected from age-related macular degeneration, neovascular age-related macular degeneration, diabetic macular edema, pathological myopia, branch retinal vein occlusion, or central retinal vein occlusion, the method comprising administering to an eye of a subject in need of such treatment at least one biologically active compound encapsulated in a matrix, wherein said matrix comprises a biodegradable, phase separated, thermoplastic poly(ether ester) multi-block copolymer [(R¹R²ₙR³)_{q}]ᵣ[(R⁴ₚR⁵R⁶ₚ)]ₛ, (PEE-MBCP) consisting of segments linked by a 1,4-butanediisocyanate chain extender, said segments being selected from the group consisting of an amorphous hydrolysable (R¹R²nR³)_{q} pre-polymer (A) segment and a semi-crystalline hydrolysable (R⁴ₚR⁵R⁶ₚ) pre-polymer (B) segment with the proviso that said multi-block copolymer comprises at least one pre-polymer (A) segment and at least one pre-polymer (B) segment, wherein

said PEE-MBCP under physiological conditions has a T_{g} of 37° C. or less and a Tₘ of 50-110° C.; and

the segments are randomly distributed over the polymer chain; and

wherein

R¹ and R³ are each

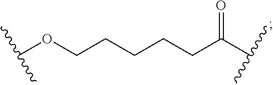 [SMILES: *OCCCCCC(*)=O]

R² is

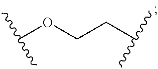 [SMILES: *CCO*]

R⁴ and R⁶ are each

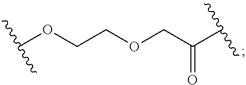 [SMILES: *OCCOCC(*)=O]

R⁵ is

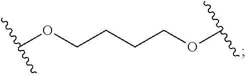 [SMILES: *OCCCCO*]

n, being the number of repeating R² moieties, is 20-120;

p, being the number of repeating R⁴ and R⁶ moieties is 10 or more;

q, being the molecular weight of the (R¹R²ₙR³) block is 1500-9000 g/mol; and

the ratio r/s is 0.1-2.5, wherein r is the weight fraction of pre-polymer (A) segment and s is the weight fraction of pre-polymer (B) segment, relative to the total amount of pre-polymer (A) and (B).

As disclosed herein, provided herein the PEE-MBCP of said method of treatment has a “q” from about 1500 to about 7000 g/mol; has a “r” from about 20 to about 70%; and has a “s” from about 30 from 80%.

As disclosed herein, the PEE-MBCP of said method of treatment has an intrinsic viscosity of 0.7-0.9 dl/g.

As disclosed herein, the PEE-MBCP of said method of treatment has an “n” from about 20 to about 70.

As disclosed herein, the PEE-MBCP of said method of treatment has an “n” from about 21 to about 46.

As disclosed herein, the PEE-MBCP of said method of treatment has a “p” from about 10 to about 15.

As disclosed herein, the PEE-MBCP of said method of treatment has a “p” from about 10 to about 13.

As disclosed herein, the PEE-MBCP of said method of treatment has a “q” from about 1550 to about 5000 g/mol.

As disclosed herein, the PEE-MBCP of said method of treatment has a “q” from about 1600 to about 4000 g/mol.

As disclosed herein, the PEE-MBCP of said method of treatment has a “r” from about 30 to about 65%.

As disclosed herein, the PEE-MBCP of said method of treatment has a “r” from about 40 to about 60%.

As disclosed herein, the PEE-MBCP of said method of treatment has a “s” from about 35 to about 70%.

As disclosed herein, the PEE-MBCP of said method of treatment has a “s” from about 40 to about 60%.

As disclosed herein, the PEE-MBCP of said method of treatment has a “n” from about 22 to about 23; has a “p” from 10.5; has a “q” from about 2000 g/mol; has a “r” from about 60%; and has a “s” from about 40%.

As disclosed herein, the PEE-MBCP of said method of treatment has a “n” from 22 to about 23; has a “p” from about 11.5; has a “q” from about 2000 g/mol; has a “r” from about 60%; and has a “s” from about 40%.

As disclosed herein, the PEE-MBCP of said method of treatment has a “n” from about 22 to about 23; has a “p” from about 12.5; has a “q” from about 2000 g/mol; has a “r” from about 60%; and has a “s” from about 40%.

As disclosed herein, the pre-polymer (B) segment of said PEE-MBCP of said method of treatment has a polydispersity index in the range of 0.6-3, such as in the range of 0.7-2, or in the range of 0.8-1.6.

As disclosed herein, the pre-polymer (B) segment of said PEE-MBCP of said method of treatment has a T_{g} of less than 0° C., preferably less than −20° C., more preferably less than −40° C.; and/or—a Tₘ in the range of 60-100° C., preferably in the range of 75-95° C.

As disclosed herein, the composition or injectable delivery system of said method of treatment is delivered in the form of microspheres, microspheres, nanoparticles, nanospheres, rods, implants, gels, coatings, films, sheets, sprays, tubes, membranes, meshes, fibres, or plugs.

As disclosed herein, the composition or injectable delivery system of said method of treatment comprises at least one biologically active compound is a non-peptide non-protein small sized drug, or a biologically active polypeptide.

As disclosed herein, the composition or injectable delivery system of said method of treatment comprises a binding protein which comprises an ankyrin repeat domain.

As disclosed herein, the composition or injectable delivery system of said method of treatment comprises a binding protein that binds VEGF-Axxx with a Kd below 10⁹ M.

As disclosed herein, the composition or injectable delivery system of said method of treatment comprises a binding protein that comprises SEQ ID NO:1.

As disclosed herein, the binding protein of said composition or injectable delivery system of said method of treatment further comprises a polyethylene glycol moiety having a molecular weight of at least 5 kDa.

As disclosed herein, the binding protein of said composition or injectable delivery system of said method of treatment is conjugated at its C-terminus via a peptide bond to a polypeptide linker and a C-terminal Cys residue, wherein the thiol of the C-terminal Cys is further conjugated to a maleimide-coupled polyethylene glycol.

As disclosed herein, the maleimide-coupled polyethylene glycol of said binding protein of said composition or injectable delivery system of said method of treatment is α-[3-(3-maleimido-1-oxopropyl)amino]propyl-ω-methoxy-polyoxyethylene.

As disclosed herein, the binding protein of said composition or injectable delivery system of said method of treatment has an N-terminal capping module of the polypeptide of SEQ ID NO:1 and comprises an Asp residue at position 5.

As disclosed herein, the multi-block copolymer of said composition or injectable delivery system of said method of treatment is in the form of a plurality of polymeric microspheres that are each not less than about 20 μm in diameter.

### EXAMPLES

### Example 1: PLGA

PLGA polymers are most often used for sustained release of drugs and have been clinically proven to be safe in the body. Even though PLGA polymers are fairly versatile, and their physiochemical properties can be tuned to accommodate different drug delivery needs, their suitability has been shown to be limited in protein delivery. Protein stability remains a major obstacle in delivering proteins with PLGA due to (1) the hydrophobic character of the polymers, (2) the formation of acidic degradation products and the accumulation of acidic degradation products in the polymer matrix leading to an in situ pH drop due to which the any encapsulated proteins may degrade and lose their biological activity. Proteins have also been shown to be (3) chemically modified through deamination or acylation within the PLGA matrix. Consequently, delivery systems made with PLGA are associated with all the issues as mentioned above including (4) protein aggregation and (5) undesirable release kinetics. Rarely do PLGA systems meet any of the above 5 criteria.

### Example 2: SynBiosys PCL05-Based Abicipar Pegol Microspheres

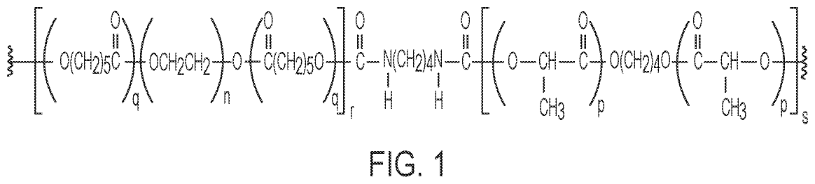

Biodegradable phase separated segmented multi-block copolymers (SynBiosys, InnoCore Technologies B.V, Groningen, The Netherlands) as disclosed in WO-A-2012/005594 and WO-A-2013/015685 have been developed to deliver peptides and proteins structurally intact and biologically active over extended periods of time up to three to six months (Stankovic et al., Eur. J. Pharm. Sci. 2013, 49(4), 578-587; Teekamp et al., Int. J. Pharm. 2017, 534(1-2), 229-236; Teekamp et al., J. Controlled Release 2018, 269(10), 258-265; Scheiner et al., ACS Omega 2019, 4(7), 11481-11492). 101361 SynBiosys multi-block copolymers are typically composed of two different blocks in which commonly used monomers including D,L-lactide, glycolide, e-caprolactone and polyethylene glycol (PEG) are copolymerized into a low molecular weight polymer (a prepolymer), which are linked together with a diisocyanate, typically 1,4-butanediisocyanate. By using two chemically and physically distinct pre-polymer blocks, such as a hydrophilic amorphous and a hydrophobic crystalline domain a phase separated segmented multi-block copolymer is obtained that provide mechanisms for long term release of drugs including peptides and proteins. The hydrophilic amorphous blocks typically contain a high content of polyethylene glycol (PEG) which leads to swelling of the multi-block copolymer under aqueous conditions. The hydrophobic crystalline blocks act as physical crosslinks. Hydrophilic phase separated segmented multi-block copolymers containing a hydrophobic poly(L-lactide)-based crystalline block (FIG. 1) are disclosed in WO-A-2013/015685. One hydrophilic phase separated segmented multi-block copolymers in particular, PCL05, was previously shown to have highly beneficial attributes in regard to protein delivery. PCLO5 (also abbreviated as 50CP10C20-LL40) is composed of a crystalline poly(L-lactide) block with a molecular weight (Me) of 4000 g/mol (abbreviated as LL40) in combination with a hydrophilic poly(ϵ-caprolactone)-PEG1000-poly(ϵ-caprolactone) block with Mₙ of 2000 g/mol (abbreviated as CP10C20) in a 50/50 weight ratio.

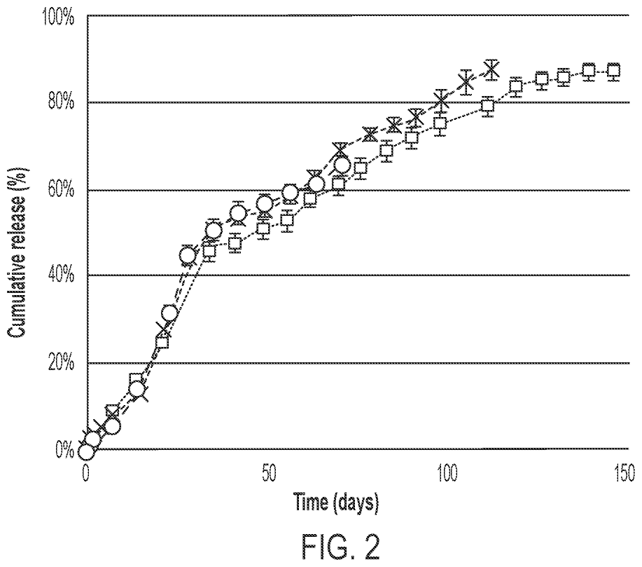

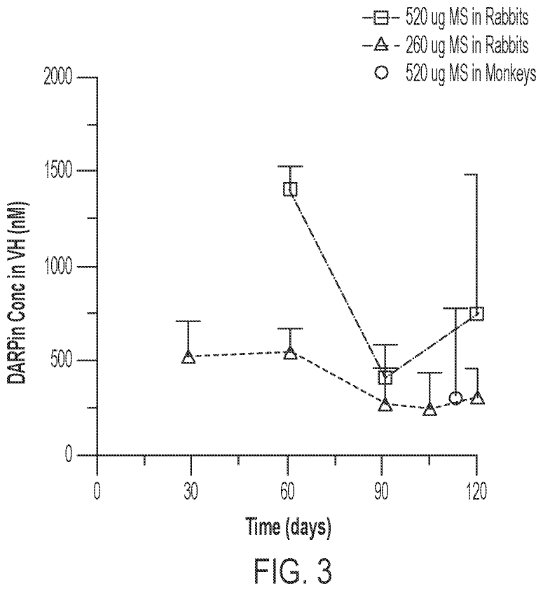

PCLO5 microspheres were shown to deliver abicipar pegol in vitro with minimal burst over a 4-month period, display minimal degradation and aggregation of protein in the delivery system and on release of the protein, and achieve therapeutic levels of protein in the vitreous for four months. FIG. 2 shows in vitro release profiles of abicipar pegol from the PCLO5 microspheres loaded with 5.2% abicipar pegol. FIG. 3 shows vitreous humor levels in rabbits and monkeys over four months achieved from an intravitreal injection of 5 or 10 mg of abicipar pegol loaded PCL05 microspheres (5.2% abicipar pegol loading) suspended in 50 pl of aqueous injection vehicle (PK14056) representing intravitreal administration of 260 μg or 520 μg of abicipar pegol.

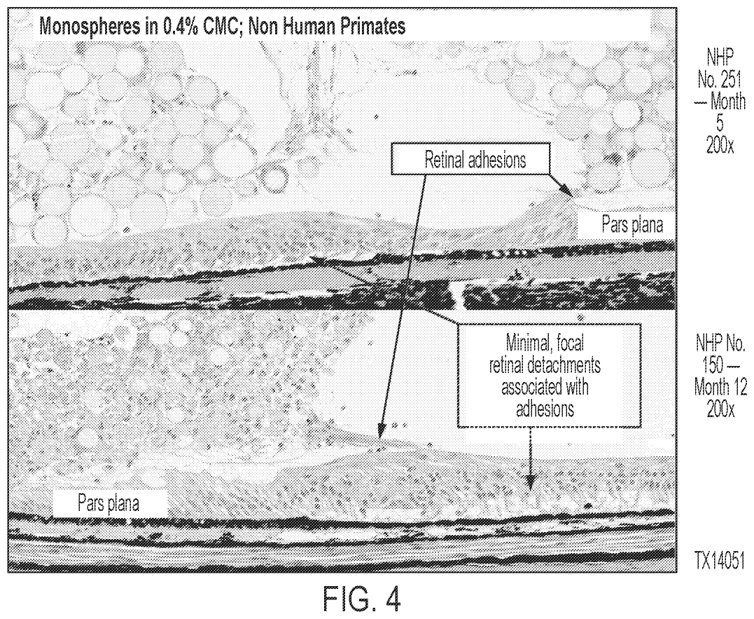

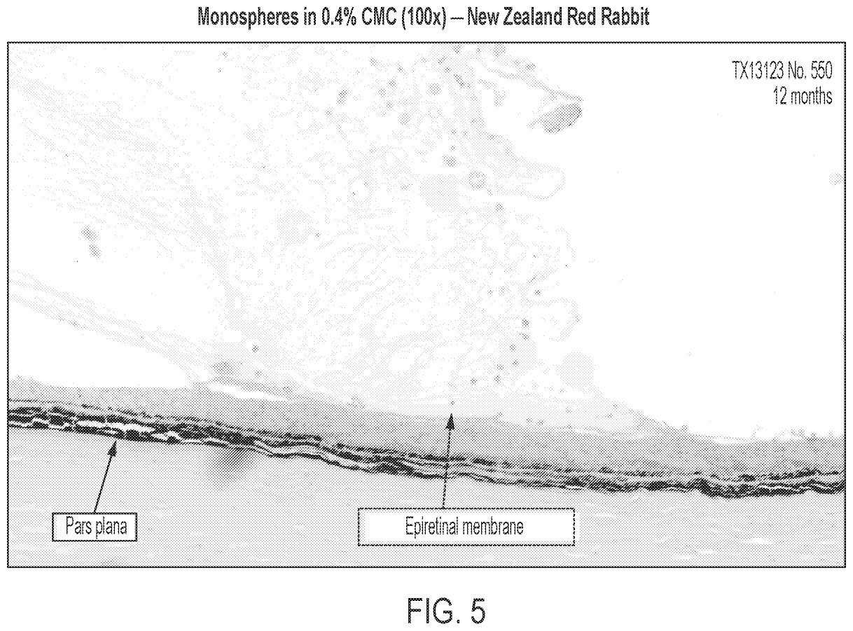

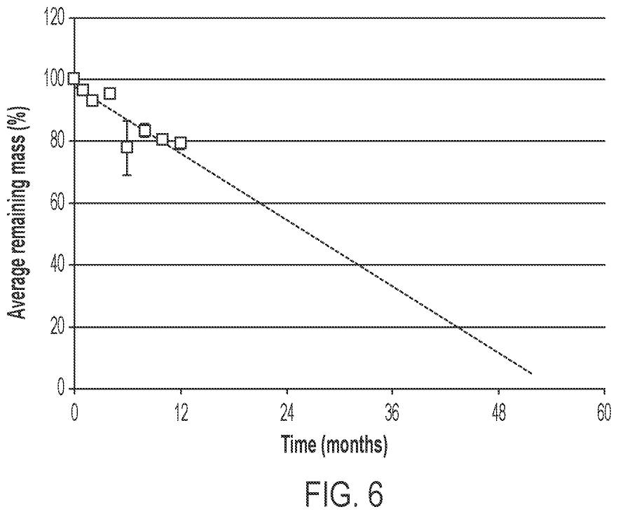

Unfortunately, the systems demonstrated a late inflammation as well as epiretinal membrane formation and retinal detachment (FIG. 4 and FIG. 5). FIG. 4 shows retinal adhesions and retinal detachments in monkeys and FIG. 5 shows epiretinal membranes observed in the NZR rabbit vitreous following administration of 10 mg of abicipar pegol loaded PCLO5 microspheres (representing 520 μg of abicipar pegol) suspended in 50 μl of aqueous injection vehicle by intravitreal injection. In particular, slow erosion of PCL05 microspheres was observed both in vivo and in vitro. Based on extrapolation of experimental data, the erosion time of the PCL0O05 microspheres is projected to be approximately 4 years in vitro (1 x PBS buffer at 37° C.) (FIG. 6) and at least 14-16 months in the rabbit vitreous. The PCL05-based abicipar pegol microspheres met three of the five requirements for an acceptable intravitreal protein delivery system.

### Example 3: Development of Faster Degrading SynBiosys-Based Abicipar Pegol Microspheres

A redesign of the SynBiosys multi-block copolymer was conducted in an attempt to reduce the erosion time of the polymer in the vitreous and improve the intravitreal tolerability. To increase its erosion rate, both the [CP10C]20 amorphous block and the crystalline LL40 block were altered. The crystalline LL40 block was altered by 1) partial replacement of L-lactide by D-lactide (L-MBCP concept), 2) use of more hydrophilic initiators for the synthesis of the crystalline L-lactide block (I-MBCP concept), 3) use of short stereo-complexed crystalline blocks composed of L-lactide and D-lactide (SC-MBCP concept); and 4) complete replacement of L-lactide by dioxanone (D-MBCP concept). The amorphous CP10C20 block was altered by changing the weight fractions and molecular weight of PEG, the length of the poly(e-caprolactone) chains and by partial replacement of c-caprolactone by DL-lactide. Finally the ratio between the amorphous and crystalline block (block ratio) were altered.

### L-MBCP Polymers

The various L-MBCP-based polymers that were synthesized are listed in Table 1. The table represents L-MBCP polymers that were prepared by chain-extending crystalline lactide-based crystalline blocks with D-lactide/L-lactide ratios of 0/100 (PCL05), 1/99, 4/96 and 7/93 mol/mol with amorphous [CP10C]20 or poly(DL-lactide-co-ϵ-caprolactone)-PEG1000-poly(DLD-lactide-co-ϵ-caprolactone) pre-polymers ([LCP1OLC]20 with DL-lactide / c-caprolactone ratios (L/C ratio) of 0/100, 5/95 and 15/85 mol/mol.

TABLE 1
Overview of L-MBCP polymers.

| | Amorphous block | | | | Crystalline block | | | |
|---|---|---|---|---|---|---|---|---|
| | | L/C | PEG | | | DL/LL | | IV |
| RCP | Type | ratio | MW | MW | Type | ratio | Block ratio | (dl/g) |
| 1446 | CP10C20 | 0/100 | 1000 | 2000 | LL40 | 0/100 | 50/50 | 0.85 |
| 1515 | CP10C20 | 0/100 | 1000 | 2000 | [DL/LL]40 | 4/96 | 50/50 | 0.98 |
| 1518 | CP10C20 | 0/100 | 1000 | 2000 | [DL/LL]40 | 1/99 | 50/50 | 1.00 |
| 1519 | CP10C20 | 0/100 | 1000 | 2000 | [DL/LL]40 | 7/93 | 50/50 | 1.12 |
| 1561 | CP10C20 | 0/100 | 1000 | 2000 | [DL/LL]40 | 7/93 | 30/70 | 0.87 |
| 1530 | LCP10LC20 | 5/95 | 1000 | 2000 | LL40 | 0/100 | 50/50 | 0.78 |
| 1532 | LCP10LC20 | 15/85 | 1000 | 2000 | LL40 | 0/100 | 50/50 | 0.73 |
| 1541 | LCP10LC20 | 5/95 | 1000 | 2000 | [DL/LL]40 | 4/96 | 50/50 | 0.96 |
| 1542 | LCP10LC20 | 15/85 | 1000 | 2000 | [DL/LL]40 | 4/96 | 50/50 | 0.90 |
| 1543 | LCP10LC20 | 15/85 | 1000 | 2000 | [DL/LL]40 | 7/93 | 50/50 | 0.85 |
| 1550 | LCP10LC20 | 5/95 | 1000 | 2000 | [DL/LL]40 | 4/96 | 30/70 | 0.88 |
| 1554 | LCP10LC20 | 15/85 | 1000 | 2000 | [DL/LL]40 | 7/93 | 30/70 | 0.92 |
| 1551 | LCP10LC20 | 15/85 | 1000 | 2000 | [DL/LL]40 | 4/96 | 30/70 | 0.91 |
| 1553 | LCP10LC20 | 5/95 | 1000 | 2000 | [DL/LL]40 | 7/93 | 30/70 | 0.81 |

### I-MBCP Polymers

To prepare more hydrophilic L-lactide-based crystalline blocks, diethylene glycol (DEG) and triethyleneglycol (TEG) were used as initiator as an alternative for 1,4-butanediol. DEG and TEG initiated LL40 pre-polymer blocks were combined with either [CP10C]20 or [LCP10LC]20. Table 2 lists the DEG and TEG-based I-MBCP polymers.

TABLE 2
Overview of I-MBCP polymers.

| | Block 1 | | | | | | | |
|---|---|---|---|---|---|---|---|---|
| | | L/C | PEG | | Block 2 | | Block | IV |
| RCP | Type | ratio | MW | MW | Type | Initiator | ratio | (dl/g) |
| 1516 | CP10C20 | 0/100 | 1000 | 2000 | LL40 | DEG | 50/50 | 0.60 |
| 1517 | CP10C20 | 0/100 | 1000 | 2000 | LL40 | TEG | 50/50 | 0.73 |
| 1548 | LCP10LC20 | 15/85 | 1000 | 2000 | LL40 | TEG | 50/50 | 0.76 |
| 1564 | CP10C20 | 0/100 | 1000 | 2000 | LL40 | TEG | 40/60 | 0.87 |

### D-MBCP Polymers

As an alternative to L-lactide-based crystalline blocks, polydioxanone was evaluated. Polydioxanone is a crystalline polyester but more hydrophilic than poly(L-lactide). Low molecular weight polydioxane-based pre-polymers were synthesized and chain-extended with CPC20 and caprolactone-PEG-based pre-polymers with varying PEG molecular weight and poly(e-caprolactone) chain lengths. Table 3 lists the various D-MBCP polymers that were prepared.

TABLE 3
Overview D-MBCP polymers.

| | Block 1 | | | | | | | |
|---|---|---|---|---|---|---|---|---|
| | | L/C | PEG | | Block 2 | | Block | IV |
| RCP | Type | ratio | MW | MW | Type | Initiator | ratio | (dl/g) |
| 1502 | CP10C20 | 0/100 | 1000 | 2000 | Poly(dioxanone) | BDO | 57/43 | 1.43 |
| 1524 | CP30C40 | 0/100 | 3000 | 4000 | Poly(dioxanone) | BDO | 50/50 | 1.20 |
| 1556 | CP15C20 | 0/100 | 1500 | 2000 | Poly(dioxanone) | BDO | 50/50 | 0.95 |
| 1557 | CP30C40 | 0/100 | 3000 | 4000 | Poly(dioxanone) | BDO | 20/80 | 0.80 |
| 15102 | CP10C16.7 | 0/100 | 1000 | 1670 | Poly(dioxanone) | BDO | 60/40 | 0.79 |
| 1567 | CP15C20 | 0/100 | 1500 | 2000 | Poly(dioxanone) | BDO | 35/65 | 0.63 |
| 15106 | CP10C12.5 | 0/100 | 1000 | 1250 | Poly(dioxanone) | BDO | 40/60 | 0.61 |
| 15102 | CP10C16.7 | 0/100 | 1000 | 1670 | Poly(dioxanone) | BDO | 60/40 | 0.79 |

### SC-MBCP Polymers

SC-MBCP polymers were obtained by chain extending amorphous pre-polymers with 50/50 wt. % mixtures of low molecular weight D-lactide pre-polymers (DL15, DL20) and L-lactide pre-polymers (LL15, LL20). D-lactide blocks and L-lactide blocks will form highly crystalline blocks via stereo-complexation. DL15/LL15 or DL20/LL20 pre-polymer mixtures were combined with CP10C20, LCP1OLC20 as well as with lower molecular weight amorphous pre-polymers composed of PEG600 (CP6C12, LCP6LC12) (Table 4).

TABLE 4
Overview of SC-MBCP polymers.

| | Block 1 | | | | | | IV |
|---|---|---|---|---|---|---|---|
| | | L/C | PEG | | Block 2 | Block | (dl/ |
| RCP | Type | ratio | MW | MW | Type | ratio | g) |
| 1332 | LCP10LC20 | 2.5/97.5 | 1000 | 2000 | SC LL15/DL15 | 50/50 | 0.56 |
| 1585 | CP10C20 | 0/100 | 1000 | 2000 | SC LL20/DL20 | 70/30 | 0.82 |
| 15117 | LCP10LC20 | 2.5/97.5 | 1000 | 2000 | SC LL15/DL15 | 50/50 | 0.84 |
| 1631 | CP6C12 | 0/100 | 600 | 1200 | SC LL15/DL15 | 80/20 | 0.88 |
| 1616 | LCP6LC12 | 2/98 | 600 | 1200 | SC LL15/DL15 | 60/40 | 0.84 |
| 1617 | LCP6LC12 | 2/98 | 600 | 1200 | SC LL15/DL15 | 70/30 | 0.86 |
| 1633 | LCP6LC12 | 2/98 | 600 | 1200 | SC LL15/DL15 | 20/80 | 0.82 |
| 1620 | LCP6LC12 | 2/98 | 600 | 1200 | SC LL20/DL20 | 70/30 | 0.80 |
| 1622 | LCP6LC12 | 2/98 | 600 | 1200 | SC LL20/DL20 | 80/20 | 0.84 |
| 1634 | LCP6LC12 | 2/98 | 600 | 1200 | SC LL20/DL20 | 90/10 | 0.90 |
| 1619 | LCP10LC20 | 2/98 | 1000 | 2000 | SC LL20/DL20 | 70/30 | 0.81 |
| 1621 | LCP10LC20 | 2/98 | 1000 | 2000 | SC LL20/DL20 | 80/20 | 0.93 |

The synthesized L-MBCP, I-MBCP, SC-MBCP and D-MBCP polymers were evaluated for their processability (particle size distribution, microscopic appearance, stickiness, absence of agglomeration, and encapsulation efficiency) into polymer-only and abicipar pegol-loaded microspheres, for the in vitro release kinetics of abicipar pegol (burst release, release duration, release kinetics and recovery) and the integrity of abicipar pegol released from the microspheres. Polymers that were well processable and that yielded abicipar pegol-loaded microspheres with acceptable encapsulation efficiency, and sustained release of intact abicipar pegol were further evaluated for their in vitro erosion kinetics.

The particle size distribution of the abicipar pegol microspheres was measured by laser diffraction. Microspheres were suspended in water until transmittance was within 70-90% and the particle size distribution of the suspension was determined within the range of 10 nm-5000 μm. The surface morphology of the abicipar pegol microspheres was evaluated by scanning electron microscopy, using a JEOL JCM-5000 Neoscope. A small amount of microspheres was adhered to carbon conductive tape and coated with gold for 3 min. The sample was imaged using a 10 kV electron beam. Abicipar pegol content was determined by dissolving abicipar pegol microspheres in DMSO, extracting abicipar pegol with 10 mM PBS and analysis of abicipar pegol concentration and purity in the supernatant by UP-SEC. UP-SEC analysis was conducted using a Waters ACQUITY UPLC Protein BEH SEC Column and a fluorescence detector (λₑₓ=280 nm, λₑₘ=350 nm). In vitro release (IVR) studies of abicipar pegol loaded microspheres were conducted in an aqueous-buffer (100 mM phosphate buffer pH 7.4+0.05 v/v % Tween 80+0.02 w/v % NaN₃) at 37° C. Samples taken at pre-determined time points until completion of release were analyzed with UP-SEC to establish the cumulative abicipar pegol release against sampling time. The in vitro erosion of non-loaded polymer-only microspheres were measured in 100 mM of phosphate buffer pH 7.4 (90-100 mg of microspheres in 10 ml). The samples were incubated at 37° C. At each sampling point, the microspheres were collected, freeze-dried and weighed.

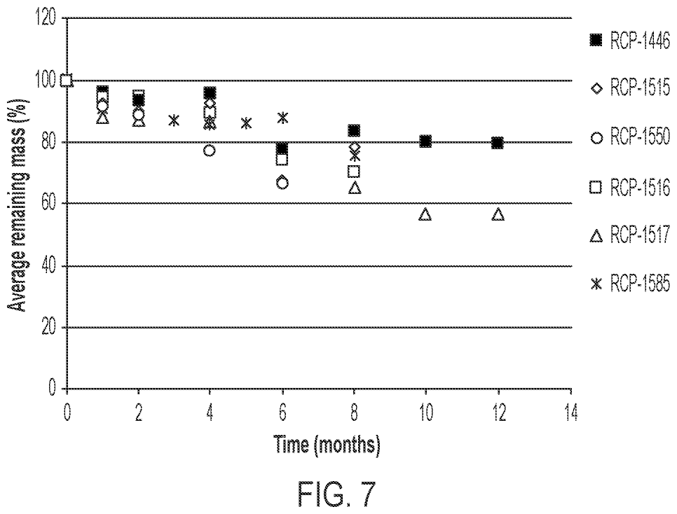

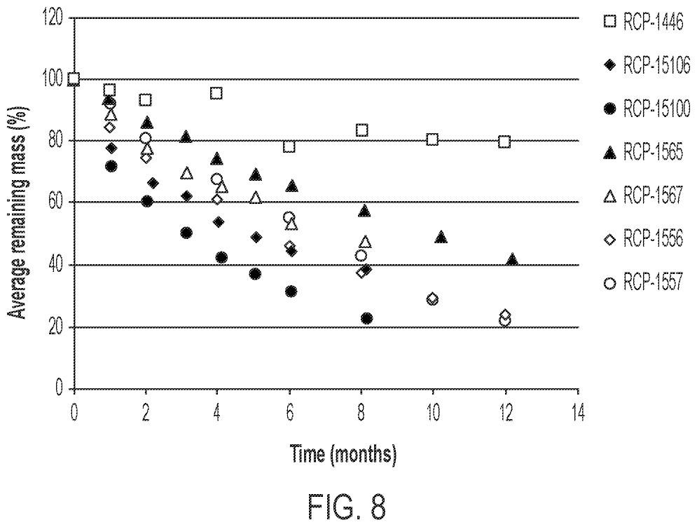

FIG. 7 shows the in vitro erosion kinetics of polymer-only microspheres prepared of the selected L-MBCP, I-MBCP and SC-MBCP polymers, whereas FIG. 8 shows the in vitro erosion kinetics of microspheres prepared of the selected D-MBCP polymers.

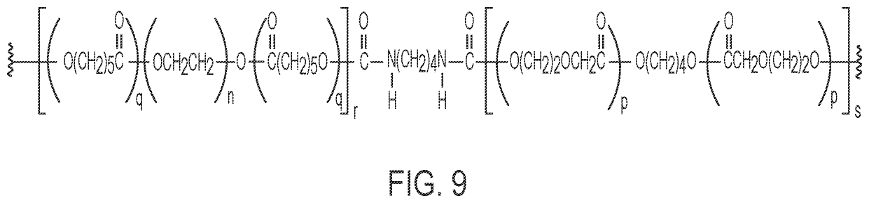

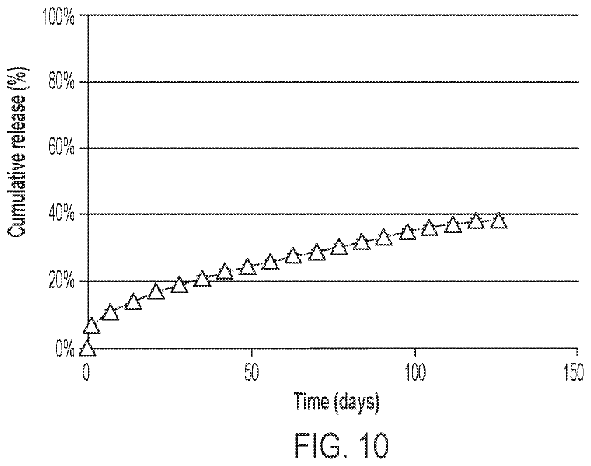

The majority of the polymers were well processable allowing the manufacturing of abicipar pegol loaded microspheres with acceptable particle size distribution and encapsulation efficiency. The polymers of the L-MBCP, I-MBCP and SC-MBCP families, however, showed very slow in vitro erosion (FIG. 7), in spite of the fact that only a few of them were shown to be promising in vitro from a protein release, and protein stability perspective. On the other hand, all multi-block copolymers based on a polydioxanone replacement of PLLA in the B block (D-MBCP polymers) were found to erode significantly faster in vitro as compared to all other multi-block copolymers. The D-MBCP polymers 50CP15C20-D25, 40CP10C12.5-D23, 60CP10C12.5-D23 and 20CP30C40-D25, however, exhibited poor in vitro release kinetics (high burst release, short release duration, low abicipar pegol recovery). A 60CP10C20-D25 D-MBCP-based multi-block copolymer (also abbreviated as PCD21) composed of a crystalline polydioxanone block in combination with a hydrophilic poly(e-caprolactone)-PEG1000-poly(e-caprolactone) block with a molecular weight (Mₑ) of 2000 g/mol in a 60/40 weight ratio (FIG. 9) was found to be most promising as it exhibited long-term in vitro release of intact abicipar pegol (FIG. 10) in combination with significantly faster in vitro erosion (FIG. 8).

### Ocular Tolerability and In Vivo Erosion Kinetics in New Zealand White Rabbits (animal study TX15024)

To allow evaluation of ocular tolerability as well as in vivo erosion, polymer-only microspheres were manufactured of a selection of the most promising multi-block copolymers and tested in vivo for their tolerability and erosion by intravitreal administration of microsphere suspensions into New Zealand White rabbits (TX15024).

TABLE 5
Polymers selected for testing of in vivo tolerability and in vivo
erosion of polymer-only microspheres (TX15024).

| Group | MBCP type | RCP | Polymer composition |
|---|---|---|---|
| 1 | L-MBCP | 1446 | 50CP10C20-LL40 (PCL05, Reference) |
| 2 | L-MBCP | 1515 | 50CP10C20-[DL/LL]40; DL/LL 4/96 |
| 3 | I-MBCP | 1564 | 40CP10C20-[TEG-LL]40 |
| 4 | D-MBCP | 1565 | 60CP10C20-D25 (PCD21) |
| 5 | I-MBCP | 1578 (1516) | 50CP10C20-[DEG-LL]40 |
| 6 | I-MBCP | 1580 (1517) | 50CP10C20-[TEG-LL]40 |
| 7 | L-MBCP | 1571 (1550) | 30LCP10LC20-[DL/LL]40; |
| | | | L/C 5/95; DL/LL 4/96 |
| 8 | D-MBCP | 1556 | 50CP15C20-D25 |

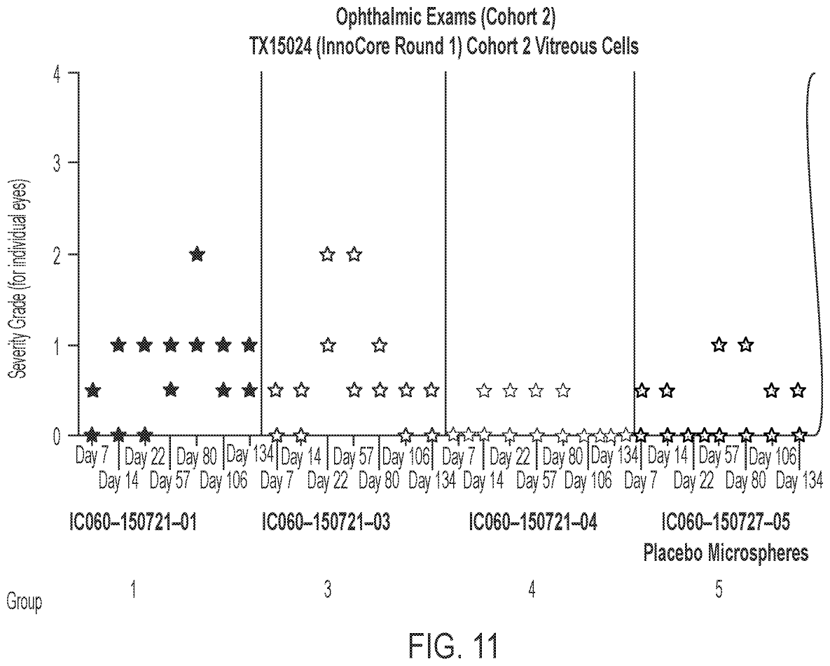

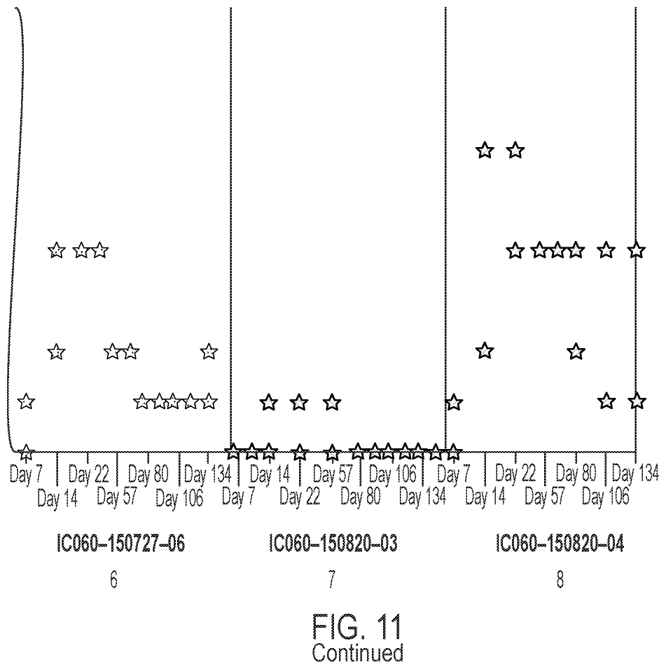

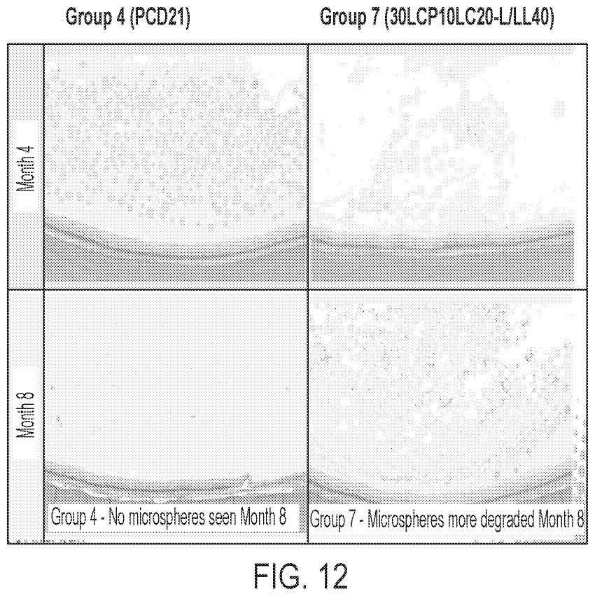

A single intravitreal injection of 10 mg of polymer-only microspheres (50 μl in PBS with 0.6% CMC) was made into the mid vitreous of rabbits. Ophthalmic observations were made up to 8 months and histology was conducted at 4 and 8 months. The microspheres of group 4 composed of the 60CP10C20-D25 dioxanone-based multi-block copolymer (PCD21, RCP-1565) were shown to be well tolerated with no retinal findings at 4 or 8 months, whereas microspheres composed of other polymers were not. FIG. 11 shows the vitreous cell score of the rabbit vitreous after administration of 10 mg of PCD21 polymer-only microspheres into the rabbit vitreous out to day 225. Additionally, compared with the observation at 4 months, histology examination at 8 months no longer revealed the presence of the 60CP10C20-D25 microspheres, suggesting severe or near complete erosion of the formulation (Table 6). FIG. 12 shows the appearance of polymer-only microspheres composed of PCD21 (Group 4) and 30LCP1OLC20-L/LL40 (Group 7) at 4 and 8 months (TX15024). Table 6 summarizes the histology results obtained with PCD21-based microspheres in New Zealand White rabbits.

TABLE 6
Polymer-only PCD21 microspheres histology results
(TX15024, NZW rabbit).

| Characteristics | Histological observations |
|---|---|
| Microsphere appearance at | Numerous intact MS ventral |
| Month 4 | retina near pars plana plus a few |
| | at back of lens. Up to 10% |
| | engulfed within macrophages. |
| Microsphere appearance at | No microspheres noted |
| Month 8 | (following section of entire eyes) |
| Retinal pathology at Month 4 | None |
| Retinal pathology at Month 8 | None |

Comparison of PCD21 with PCL05-Based Abicipar Pegol Microspheres

Overall, PCD21-based microspheres were found to be well processable into abicipar pegol loaded microspheres yielding microspheres with comparable abicipar pegol loading as well as in vitro release profiles but with significantly improved ocular tolerability and in vivo erosion characteristics as compared to PCL05-based microspheres (Table 7). The loading of abicipar pegol at 5-6% w/w is not expected to have a significant impact on either ocular tolerability or in vivo erosion. Based on the combination of in vitro results (in vitro release duration, protein purity) and in vivo erosion and ocular tolerability, the poly(dioxanone) based multi-block copolymer was selected for further development and optimization of abicipar pegol sustained release microspheres.

TABLE 7
Comparison of abicipar pegol loaded PCD21 (60CP10C20-D25)
microspheres with PCL05 (50CL10C20-LL40)-based
abicipar pegol microspheres.

| Parameter | PCL05 | PCD21 |
|---|---|---|
| Polymer grade | 50CL10C20-LL40 | 60CP10C20-D25 |
| Abicipar pegol load | 5.2% | 5-6% |
| Release duration in | 4 months | 4-5 months |
| vitro | | |
| Average daily dose | 3.4 μg | 3-4 μg |
| (for 10 mg | | |
| microspheres in | | |
| vitro | | |
| Polymer lifetime (in | 2-4 years | ~16 months |
| vitro) | | |
| Estimated polymer | At least 16 | 7-9 months in |
| lifetime (in vivo) | months in | NZR and DB |
| | rabbit; likely 2 to | rabbits; ~16 |
| | 4 years in | months in |
| | primates | primates |

### Sustained Efficacy and Bioerosion of Abicipar Pegol Loaded PCD21 Microspheres in the Eyes of New Zealand Red Rabbits

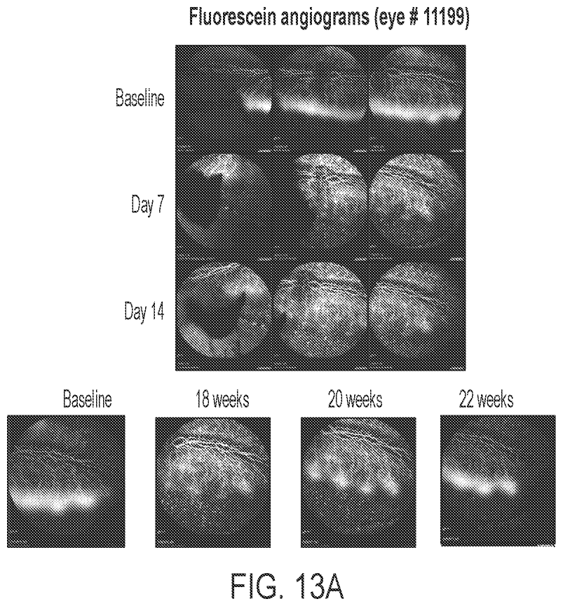

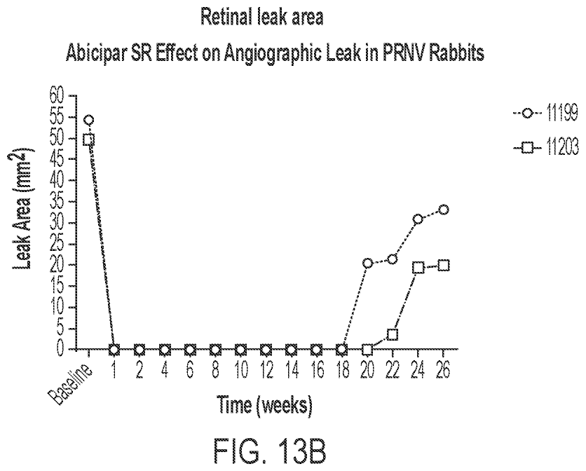

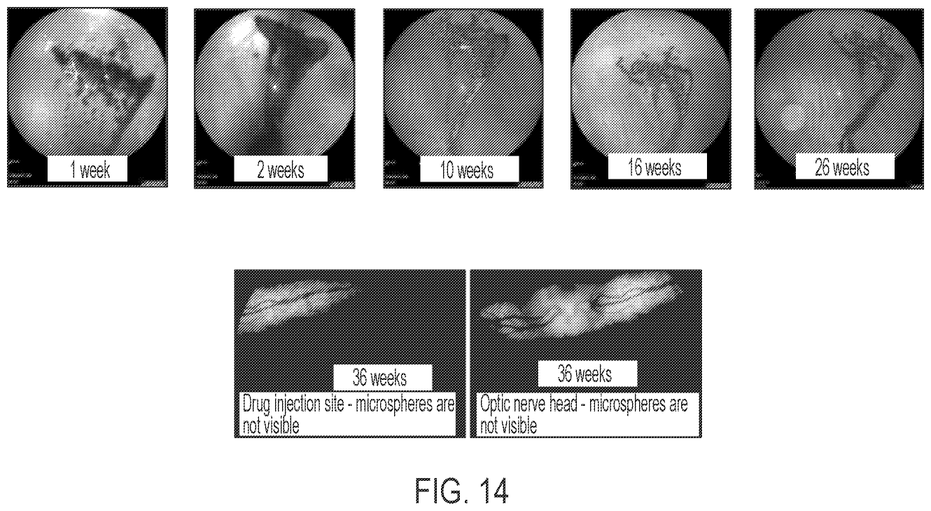

A rabbit model of persistent retinal vascular leak was used to examine the duration of efficacy of abicipar pegol from its PCD21 microsphere formulation. Two New Zealand Red rabbit eyes were injected intravitreally with a single 10 mg dose of PCD21 microspheres loaded with 4 w/w % of abicipar pegol (50 μl of microsphere suspension in PBS with 0.6% CMC) and retinal leakage was examined. The results showed a quick onset of retinal leak inhibition as early as one week and the inhibition continued for 18-20 weeks before the leak partially returned (FIGS. 13A-B). In the same study, the visible mass of the formulation in the vitreous was examined by imaging and by 36 weeks the microspheres were no longer visible either near injection site or optic nerve head (FIG. 14), suggesting the erosion of the bulk of the formulation.

### Sustained Efficacy and Bioerosion of AbiciparPegol Loaded PCD21 Microspheres in the Eyes of Dutch Belted Rabbits

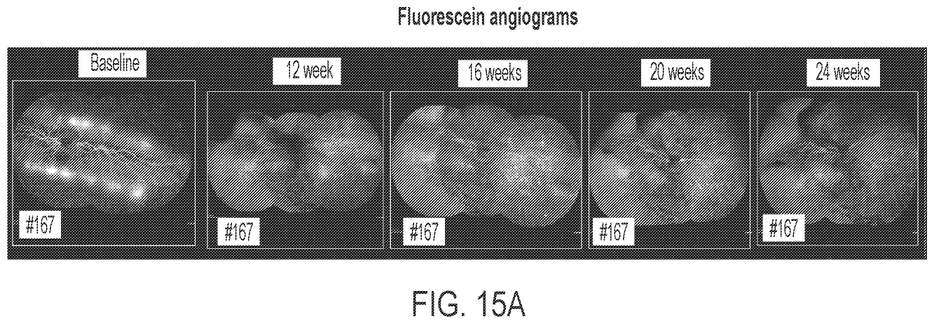

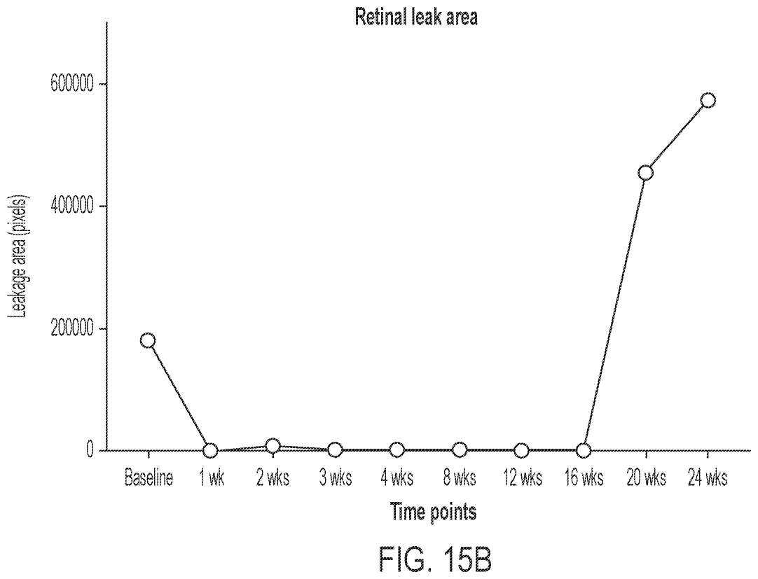

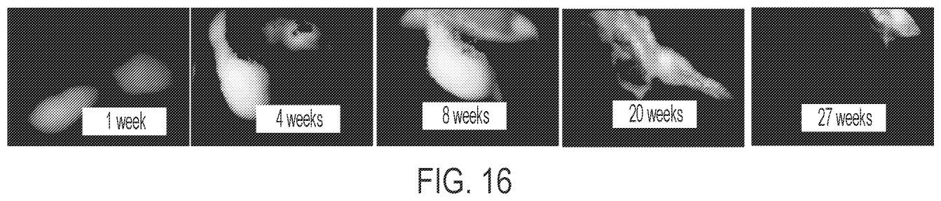

A similar study was conducted as above using a Dutch Belted rabbit as a model for persistent retinal vascular leak. Complete retinal leak suppression for at least 16 weeks has been demonstrated with one eye following a single dose of 10 mg of PCD21 microspheres loaded with 4 w/w % of abicipar pegol (50 μl of microsphere suspension in PBS with 0.6% CMC) (FIGS. 15A-B). Very little of the microspheres was visible after 27 weeks by color fundus imaging (FIG. 16).

### Ocular Tolerability of Abicipar Pegol Loaded PCD21 Microspheres in Primate Eyes (Animal Study TX16015)

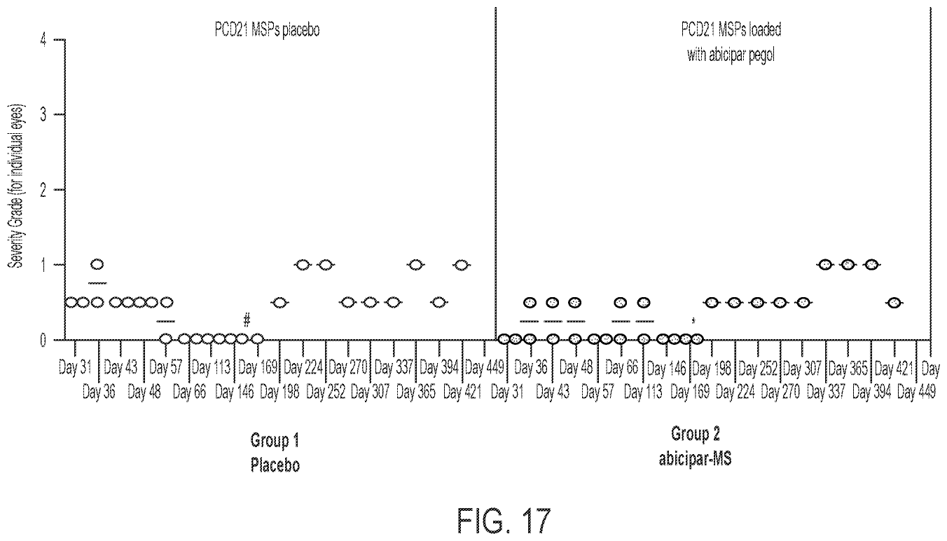

Tolerability of both placebo and abicipar loaded PCD21 microspheres (4 w/w%) was demonstrated in Cynomolgus monkeys. A single dose of 10 mg of either placebo or abicipar pegol loaded PCD21 microspheres (50 μl of microsphere suspension in PBS with 0.6% carboxymethylcellulose (CMC)) was injected into monkey vitreous and the animals were examined with ophthalmic observations up to 16 months following nearly full erosion of the microspheres. Histopathology examination was conducted at 5, 11 and 16 months. For both formulation groups, ophthalmic observations indicated no adverse findings (FIG. 17) and histopathology showed that all examined eyes were within normal limits.

### In Civo Pharmacokinetics of Abicipar Pegol-Loaded PCD21 Microspheres following Intravitreal Injection in Primate Eyes (study PK16031)

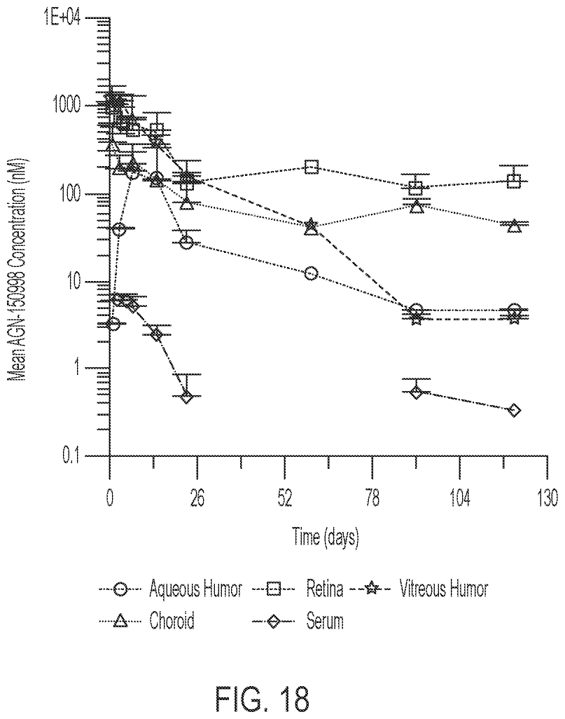

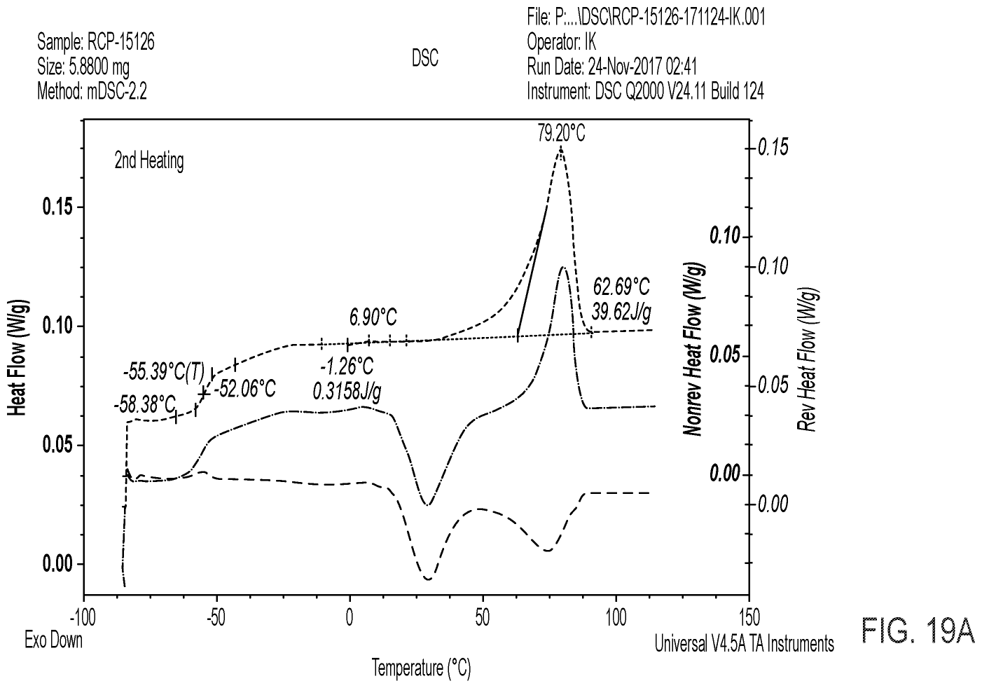

Furthermore, sustained delivery of abicipar pegol over 4 months in target ocular tissues was demonstrated with PCD21 microspheres in primate eyes. Cynomolgus monkeys were treated with a single intravitreal injection of 10 mg of PCD21 microspheres loaded with 4 w/w % of abicipar pegol (50 μl of microsphere suspension in PBS with 0.6% CMC) and the concentrations of abicipar pegol in target ocular tissues as well as serum were determined. The results indicated rapid absorption of abicipar pegol into the retina and choroid, and sustained concentration of abicipar pegol near 140 nM in the retina up to 4 months post dose with minimal anterior or systemic exposure (Table 8 and FIG. 18).

TABLE 8
Abicipar Pegol Pharmacokinetic Parameters (PK16031,
Cynomolgus monkeys)

| | | | | | | AUC₀₋ₗₐₛₜ | | | |
|---|---|---|---|---|---|---|---|---|---|
| | Cₘₐₓ (nM) | | Tₘₐₓ | Cₗₐₛₜ | Tₗₐₛₜ | (nmol · hr/l) | | AUC_{0-inf} | T_{1/2} |
| Matrix | Mean | SE | (day) | (nM) | (day) | Mean | SE | (nmol · hr/l) | (day) |
| Retina | 1020 | 310 | 1 | 139 | 120 | 25600 | 1800 | NC | NC |
| Choroid | 368 | 121 | 1 | 43.2 | 120 | 9500 | 780 | NC | NC |
| Vitreous Humor | 1120 | 130 | 3 | 3.71 | 120 | 16900 | 2000 | 17000 | 14.4 |
| Aqueous Humor | 177 | 91 | 7 | 4.58 | 120 | 3490 | 890 | 3720 | 35.0 |
| Serum | 6.25 | 0.31 | 3 | 0.341 | 120 | 102 | 8 | 121 | NC |

NC = not calculable;
SE = standard error

### Example 4: Synthesis and Characterization of Polydioxanone-Based Multi-Block Copolymers

This example describes the synthesis and characterization of [poly(ϵ-caprolactone)-co-PEG-co-poly(ϵ-caprolactone)]—b—[poly(dioxanone)] multi-block copolymers. Poly(ϵ-caprolactone)-co-PEG1000-co-poly(ϵ-caprolactone) pre-polymer with a target Mₙ of 2000 g/mol (abbreviated as ppCP10C20) was prepared by ring-opening polymerization of c-caprolactone using polyethylene glycol with a molecular weight of 1000 g/mol (PEG1000) as initiator. 500.9 g (2.00 mol) of PEG1000 (Ineos) was weighed into a three-necked bottle under nitrogen atmosphere and dried at 90° C. for at least 16 h under reduced pressure. c-Caprolactone (Acros

Organics) was dried and distilled over CaH2 under reduced pressure and stored under a nitrogen atmosphere. 495.9 g (4.34 mol) of c-caprolactone was added to the PEG under nitrogen atmosphere and the mixture was heated to 160° C. 140.1 mg of stannous octoate was added and the mixture was magnetically stirred and reacted at 160° C. during 73 h. ¹H-NMR showed ˜100% monomer conversion. Molecular weight as determined by ¹H-NMR was 1980 g/mol.

Poly(c-caprolactone)-co-PEG1500-co-poly(c-caprolactone) pre-polymer with a target Mₙ of 2000 g/mol (abbreviated as ppCP15C20) and poly(c-caprolactone-co-PEG3000-co-poly(ϵ-caprolactone) pre-polymer with a target Mₙ of 4000 g/mol (abbreviated as ppCP30C40) were prepared similarly by ring-opening polymerization using either PEG with a molecular weight of 1500 g/mol (PEG1500) or 3000 g/mol (PEG3000) as initiator. Experimental details and results obtained for synthesis of poly(ϵ-caprolactone-co-PEG-co-poly(ϵ-caprolactone) pre-polymers are listed in Table 9.

TABLE 9
Experimental details and results obtained for synthesis
poly(ε-caprolactone-co-PEG-co-poly(ε-caprolactone) pre-polymers.

| Pre- | Target | PEG | | | Stannous | Con- | Mₙ* |
|---|---|---|---|---|---|---|---|
| polymer | Mₙ | MW | CL | PEG | octoate | version | (g/ |
| A | (g/mol) | (g/mol) | (g) | (g) | (mg) | % | mol) |
| ppCP10C20 | 2000 | 1000 | 495.9 | 500.9 | 140.1 | 100% | 1980 |
| ppCP15C20 | 2000 | 1500 | 49.00 | 152.6 | 31.3 | 97.1% | 2000 |
| ppCP30C40 | 4000 | 3000 | 61.22 | 183.5 | 25.1 | 99.4% | 3970 |

Mₙ* calculated by 1H NMR

Poly(p-dioxanone) pre-polymer with different molecular weights were synthesized in the bulk by 1,4-butanediol (BDO) initiated ring-opening polymerization. BDO (Acros Organics) and p-dioxanone monomer (PDO, ≥99.5% pure, HBCChem) were distilled over CaH₂ under reduced pressure and stored under nitrogen atmosphere until further use. PDO was molten and introduced into a three necked bottle under nitrogen atmosphere. Then BDO was added to the PDO under nitrogen atmosphere. The mixture was heated to 80° C. giving a clear molten fluid. Stannous octoate (Sigma-Aldrich) was added as a solution in dioxane (Acros, dried and distilled) at a monomer catalyst ratio of 23 000 to 33 000, starting the ring-opening polymerization. The mixture was mechanically stirred at 80° C. for 25 hours. Upon solidification of poly(dioxanone) stirring was stopped. In the solid state polymerization continued and conversion increased to the targeted 80-90%. Table 10 lists the amounts of PDO monomers, BDO initiator, and stannous octoate catalyst used for the synthesis of polydioxanone pre-polymers with different molecular weight. Samples were taken from the bulk of the solidified polymer (N=3, different positions), mixed together to have one combined sample and analysed by ¹H-NMR as to determine the average conversion and molecular weight of the polymers. Polymerization was continued until conversion was >80% and varied from 80.0 to 92.7%. The number averaged molecular weights of the so-prepared poly(dioxanone) polymers (ppDxx) varied from 1783-2806 g/mol. ppDxx pre-polymers were not isolated, but left in the reactor until further use.

TABLE 10
Experimental details and results obtained for synthesis
poly(dioxanone) pre-polymers.

| Pre- | | Target | | | Stannous | Con- | Mₙ* |
|---|---|---|---|---|---|---|---|
| polymer | Batch | Mₙ | PDO | BDO | octoate | version | (g/ |
| A | nr | (g/mol) | (g) | (g) | (mg) | % | mol) |
| ppD20 | 1505 | 2000 | 59.13 | 2.21 | 7.7 | 80.0 | 1783 |
| ppD23 | 1551 | 2300 | 45.26 | 1.57 | 76.6 | 85.6 | 2043 |
| ppD25 | 1542 | 2500 | 182.64 | 6.24 | 232.4 | 92.7 | 2401 |
| ppD28 | 1716 | 2800 | 189.25 | 5.30 | 19.8 | 86.9 | 2806 |

Mₙ* calculated by ¹H-NMR

[Poly(ϵ-caprolactone)-co-PEG-co-poly(ϵ-caprolactone)]—b—[poly(dioxanone)] multi-block copolymers with various block ratios were prepared by chain-extension of ppDxx pre-polymer with ppCP10C20, ppCP15C20 or ppCP30C40 pre-polymers using 1,4-butanediisocyanate as a chain extender. First a ppDxx pre-polymer was prepared in situ in a three neck flask as described above where after the required amount of ppCP10C20, ppCP15C20 or ppCP30C40 pre-polymer prepared as described above was added. Water-free p-dioxane (Acros Organics, distilled and fractionated under reduced pressure in a modified rotary evaporator setup) was pumped into the three neck flask until a polymer concentration of 30 wt. % was reached. The flask was heated to 80° C. to dissolve the pre-polymers. 0.990 Equivalents (with respect to the pre-polymer hydroxyl groups) of 1,4-butanediisocyanate (Bayer, distilled under reduced pressure) was added. Additional stannous octoate was added to increase its total content to 45 ppm and the reaction mixture was stirred magnetically until the desired viscosity was obtained, where after distilled p-dioxane containing 20 wt. % water was added to quench unreacted isocyanate groups and stop the reaction. Stirring was continued for an additional 30 minutes. The reaction mixture was further diluted with p-dioxane to a polymer concentration of 10 wt. %, cooled to room temperature, poured into a tray and frozen at -18° C. p-Dioxane was removed from the frozen reaction solution under reduced pressure in a vacuum oven at 30° C. Table 11 lists the experimental details of the various [poly(ϵ-caprolactone)-co-PEG-co-poly(ϵ-cap rolactone)]—b—[poly(dioxanone)] multi-block copolymers.

TABLE 11
Synthesis details of [poly(ε-caprolactone)-co-PEG-co-
poly(ε-caprolactone)]-b-[poly(dioxanone)] multi-block copolymers.

| | | | Mₙ | | Mₙ | |
|---|---|---|---|---|---|---|
| | | ppDxx | ppDxx | ppCPxxCyy | ppCPxxCyy | BDI |
| Grade | RCP | (g) | (g/mol) | (g) | (g/mol) | (g) |
| 54CP10C20-D18 | 1510 | 48.74 | 1783 | 48.89 | 2000 | 6.7745 |
| 60CP10C20-D23 | 15126 | 39.28 | 2260 | 59.11 | 2041 | 6.4166 |
| 30CP15C20-D24 | 1567 | 53.57 | 2437 | 28.96 | 2000 | 5.1712 |
| 50CP15C20-D23 | 15125 | 49.89 | 2294 | 50.03 | 2000 | 6.4685 |
| 50CP15C20-D25 | 1579 | 50.58 | 2547 | 49.70 | 2000 | 6.2321 |
| 50CP30C40-D28 | 1524 | 59.11 | 2790 | 54.90 | 3091 | 4.8497 |

Polymers were stored in a sealed package at -18° C. and analyzed for polymer composition (¹H-NMR), intrinsic viscosity, residual p-dioxane content (gas chromatography) and thermal properties (mDSC).

¹H-NMR was performed on a Bruker Avance DRX 500 MHz NMR spectrometer (BAV-500) equipped with a Bruker Automatic Sample Changer (BACS 60) (Varian) operating at 500 MHz. The d₁ waiting time was set to 20 s, and the number of scans was 16. Spectra were recorded from 0 to 14 ppm.

Conversion was determined from ¹H-NMR, pre-polymer Mₙ was determined from in weights and ¹H-NMR. ¹H-NMR samples were prepared by adding 1 ml of deuterated chloroform to 10 mg of polymer.

Intrinsic viscosity was measured using an Ubbelohde Viscosimeter (DIN), type OC, Si Analytics supplied with a Si Analytics Viscosimeter including a water bath. The measurements were performed in chloroform at 25° C. The polymer concentration in chloroform was such that the relative viscosity was in the range of 1.2-2.0.

p-Dioxane content was determined using a GC-FID headspace method. Measurements were performed on a GC-FID Combi Sampler supplied with an Agilent Column, DB-624/30 m/0.53 mm. Samples were prepared in DMSO (dimethylsulphoxide). p-Dioxane content was determined using p-dioxane calibration standards.

Modulated differential scanning calorimetry (MDSC) was used to determine the thermal behavior of the multi-block copolymers using a Q2000 MDSC (TA instruments, Ghent, Belgium). About 5-10 mg of dry material was accurately weighed and heated under a nitrogen atmosphere from −85° C. to 120° C. at a heating rate of 2° C./min and a modulation amplitude of +/−0.42° C. every 80 seconds. The glass transition temperature (T_{g}, midpoint) was determined using the reversed heat flow curve, while the melting temperature (maximum of endothermic peak, Tₘ) and the melting enthalpy (ΔHₘ), which was calculated from the surface area of the melting endotherm, were determined using the total heat flow curve. Temperature and enthalpy were calibrated with an indium standard. 101671 Table 12 shows the collected analysis results regarding the actual composition, intrinsic viscosity and residual dioxane of the multi-block copolymers. The actual composition as determined by ¹H-NMR from D/P and C/P molar ratios resembled the target composition well. The intrinsic viscosity of the polymers varied between 0.54 and 1.20 dl/g. Residual dioxane contents were very low indicating effective removal thereof by vacuum-drying.

TABLE 12
Collected results regarding the chemical composition, intrinsic
viscosity and residual dioxane content of multi-block copolymers

| | | Molar CL/P ratio | | Molar D/P ratio | | | Dioxane |
|---|---|---|---|---|---|---|---|
| Grade | RCP | in- weight | ¹H- NMR | in- weight | ¹H- NMR | IV (dl/g) | content (ppm) |
| 54CP10C20-D18 | 1510 | 8.6 | 8.7 | 16.6 | 18.8 | 0.54 | <88 |
| 60CP10C20-D23 | 15126 | 9.0 | 8.8 | 11.5 | 12.8 | 0.98 | <92 |
| 30CP15C20-D24 | 1567 | 4.3 | 4.4 | 35.1 | 35.8 | 0.63 | <104 |
| 50CP15C20-D23 | 15125 | 4.3 | 4.2 | 17.2 | 18.9 | 1.13 | <92 |
| 50CP15C20-D25 | 1579 | 4.2 | 4.2 | 20.8 | 19.3 | 0.94 | <102 |
| 50CP30C40-D28 | 1524 | 8.0 | 8.0 | 37.7 | 41.0 | 1.20 | <110 |

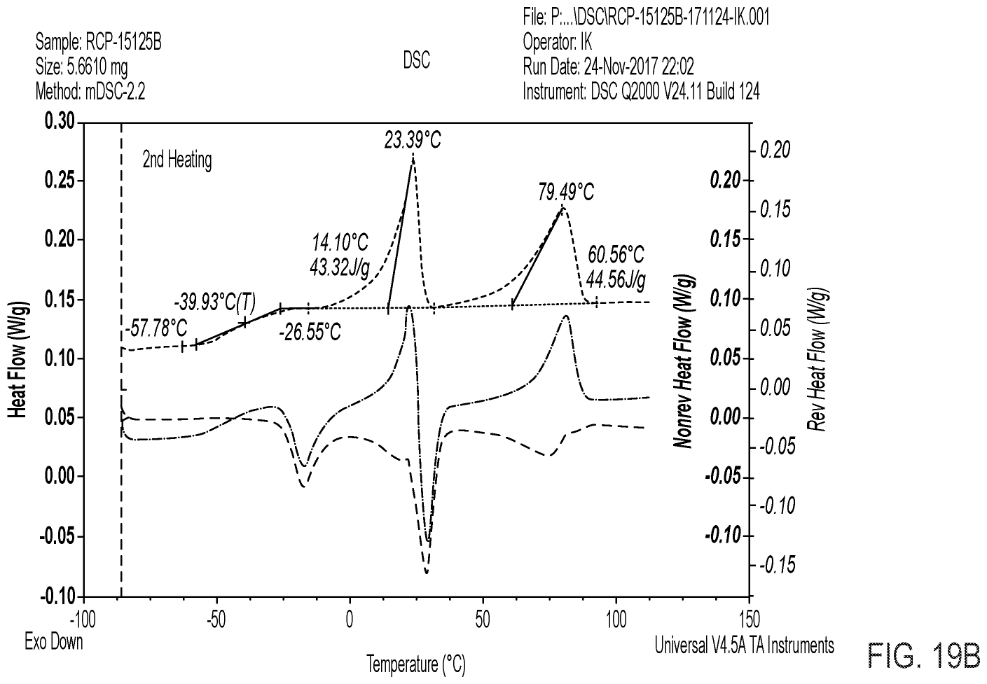

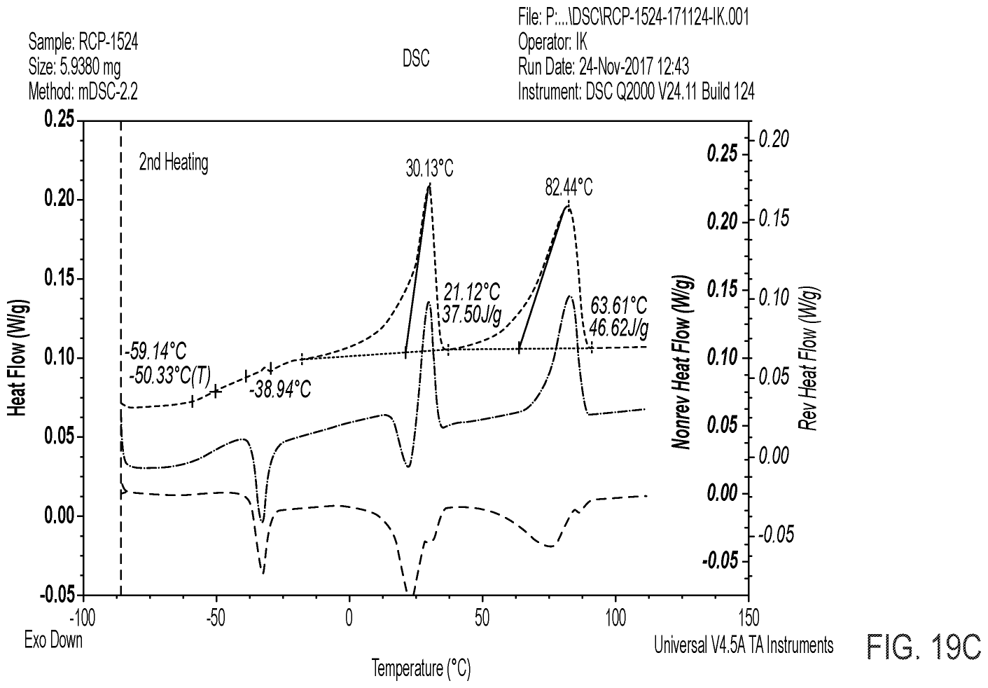

The multi-block copolymers were analysed for their thermal properties to confirm their phase separated morphology (Table 13). FIGS. 19A-C shows typical DSC thermograms of 60CP10C20-D23 (RCP 1512613 Plate A), 50CP15C20-D23 (RCP 15125 — Plate B) and 50CP30C40-D28 (RCP 1524 — Plate C) multi-block copolymers. All multi-block copolymers exhibited a melting temperature (T_{m,2}) at approximately 80° C., due to melting of the dioxanone segment. The melting enthalpy (ΔH_{m,2}) varied between 37 to 66 J/g. Additionally, in PEG1500-containing 50CP15C20-D23 and PEG3000-containing 50CP30C40-D28 a melting peak was found around 20 to 30° C. due to melting of the PEG-rich phase. The glass transition temperature (T_{g}) of the multi-block copolymers is in general in between that of the two pre-polymers, indicating phase mixing of the amorphous pre-polymer with the amorphous content of the semi crystalline pre-polymer.

TABLE 13
Thermal characteristics of multi-block copolymers

| | | T_{g} | T_{m,1} | ΔH_{m,1} (Tₘ₁) | T_{m,2} | ΔH_{m,2} |
|---|---|---|---|---|---|---|
| RCP | Grade | (° C.) | (° C.) | (J/g) | (° C.) | (J/g) |
| 1510 | 54CP10C20-D18 | −61 | N.D. | N.D | 72.0 | 37.0 |
| 15126 | 60CP10C20-D23 | −55.4 | 6.9 | 0.3 | 79.2 | 39.6 |
| 1567 | 30CP15C20-D24 | −53 | 11 | N.D. | 84.0 | 66.0 |
| 15125 | 50CP15C20-D23 | −39.9 | 23.4 | 43.3 | 79.5 | 44.6 |
| 1579 | 50CP15C20-D25 | −48.6 | 23.8 | 41.7 | 88.1 | 65.2 |
| 1524 | 50CP30C40-D28 | −50.3 | 30.1 | 37.5 | 82.4 | 46.6 |

N.D.: not detected

### Example 5: Preparation and Characterization of Abicipar Pegol Loaded Microspheres

Microspheres with a target abicipar pegol loading of 5-5.5 wt. % were prepared of multi-block copolymers described in example 4 at a scale of 2.5 g by solvent extraction/evaporation using a W1/O/W2 water-in-oil-in-water double emulsion-based membrane emulsification process. The multi-block copolymer was dissolved in dichloromethane to a concentration of 10 or 15.wt. % and filtered over a 0.2 μm PTFE filter. An aqueous solution of abicipar pegol (150 mg/ml) was emulsified with the polymer solution to obtain a primary emulsion. The primary emulsion was pumped via a membrane with 20 gm pores and into a vessel with an aqueous extraction medium containing 4.0 wt. % PVA and 5 wt. % NaCl to form a secondary emulsion. The secondary emulsion was stirred for 3 hours at room temperature to remove DCM by solvent extraction/evaporation. The resulting microspheres were collected on a 5 gm membrane filter and washed three times with 250 ml of ultrapure water containing 0.05 wt. % of Tween® 80 and three times with 250 ml of ultrapure water. Finally, the microspheres were lyophilised.

Abicipar pegol loaded microspheres were analyzed for particle size, microscopic appearance (SEM), abicipar pegol content and abicipar pegol release kinetics according to the methods described in Example 3.

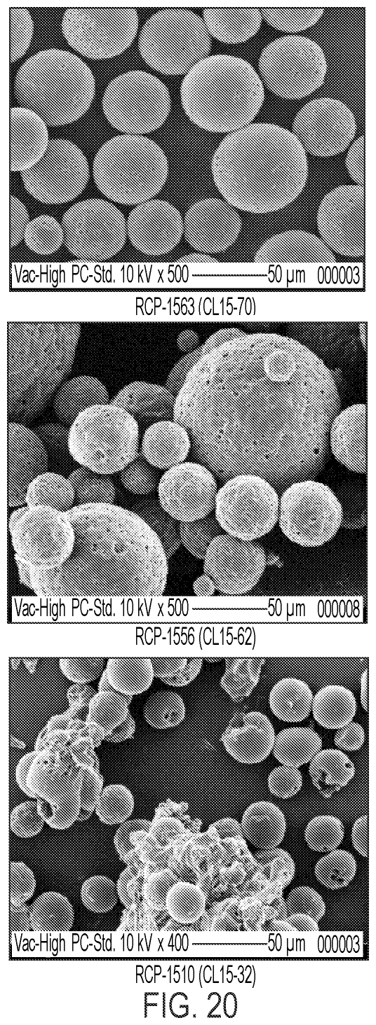

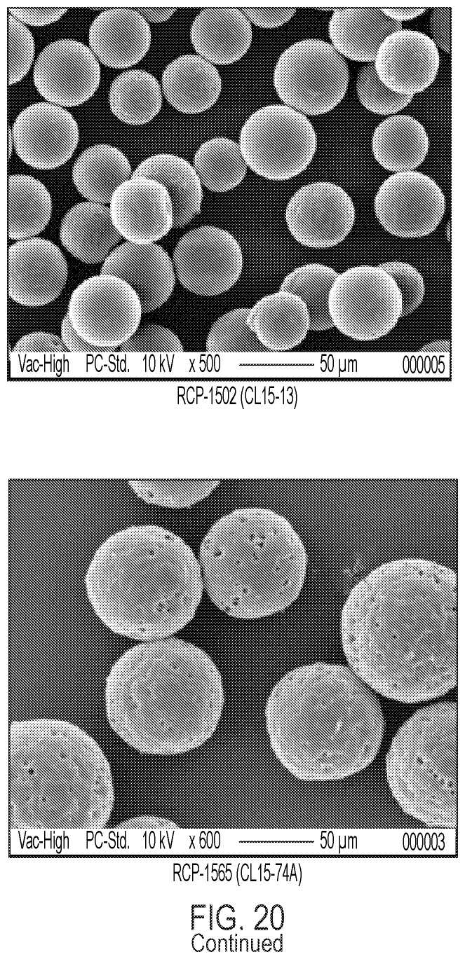

All polymers, except for RCP-1510 (54CP10C20-D18) and RCP-1562 (70CP10C20-D25), were well processable yielding abicipar pegol-loaded microspheres with an average particle size varying from 44 to 73 gm and abicipar pegol loadings varying from 1.1 to 4.6 wt. % (encapsulation efficiency (EE) ranging from 22% to 86%) (Table 14). The SEM pictures in FIG. 20 show extensive particle agglomeration and large polymer lumps for abicipar pegol loaded microspheres prepared of RCP-1510 and RCP-1562.

Abicipar pegol-loaded microspheres prepared of the other polymers did not show any agglomeration but instead had either a smooth or microporous surface morphology.

TABLE 14
Average particle size actual abicipar pegol loading,
encapsulation efficiency and burst release of abicipar pegol microspheres
prepared of different multi-block copolymers.

| | | | | Abicipar | | |
|---|---|---|---|---|---|---|
| | | | Particle | pegol | | Burst |
| | Polymer | | size | loading | EE | release |
| MSP lot # | grade | RCP | (D₅₀, μm) | (wt %) | (%) | (%) |
| CL15-70 | 30CP15C20-D25 | 1563 | 57 | 3.6 | 69 | 55 |
| CL15-62 | 50CP15C20-D23 | 1556 | 73 | 1.1 | 22 | 16 |
| CL15-32 | 54CP10C20-D18 | 1510 | 45 | 1.9 | 37 | 12 |
| CL15-13 | 57CP10C20-D28 | 1502 | 44 | 4.6 | 86 | 2 |
| CL15-74A | 60CP10C20-D25 | 1565 | 64 | 2.8 | 51 | 26 |

### In Vitro Release of Abicipar Pegol from Microspheres Composed of polydioxanone-based Multi-Block Copolymers

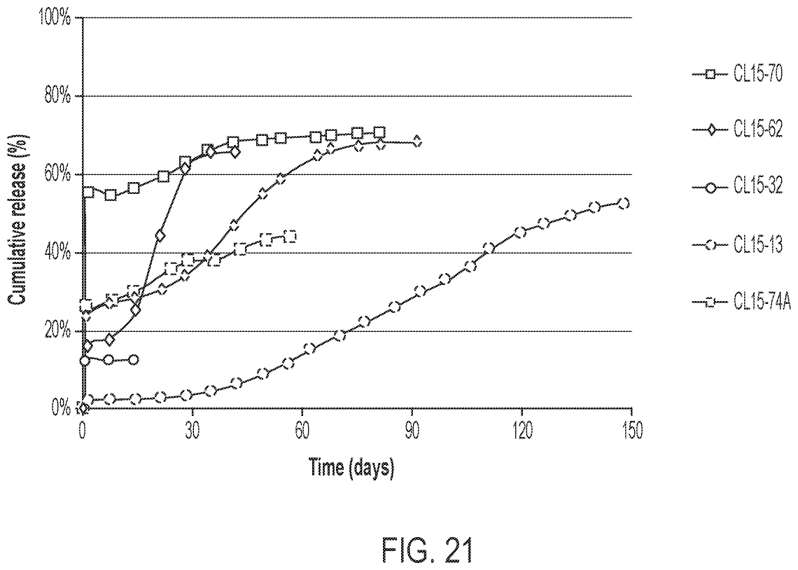

FIG. 21 shows the in vitro release kinetics for the various abicipar pegol loaded microspheres. Abicipar pegol loaded microspheres composed of 30CP15C20-D25 and 50CP15C20-D23 exhibited relatively short release duration with abicipar pegol completely released within 4 to 8 weeks. The fast release of abicipar pegol is attributed to the higher molecular weight of PEG (1500 g/mole) used in RCP-1563 and RCP-1556.

Abicipar pegol loaded microspheres (CL15-13) composed of 57CP10C20-D23 showed promising release kinetics with sustained release of abicipar pegol for more than 20 weeks. Despite its high burst release of >20%, CL15-74A, composed of a polymer (60CP10C20-D25) with similar composition as used in CL15-13, also showed promising sustained release kinetics.

### Example 6: In Vitro Erosion Kinetics of Polymer-only Microspheres Composed of Polydioxanone-Based Multi-Block Copolymers

Due to the phase-separated morphology of the multi-block copolymers, the composition of the blocks significantly affects the overall erosion kinetics of the multi-block copolymers. The content and molecular weight of PEG as well as the length of the poly(e-caprolactone) chains of the hydrophilic pre-polymer segment (A) and the molecular weight (Mₙ) of the crystalline poly(dioxanone) pre-polymer segment (B) are considered the most critical parameters for the overall erosion kinetics of the resulting multi-block copolymer. The synthesis of the polymers that were examined for their in vitro erosion kinetics was described in Example 4. The composition and relevant physicochemical characteristics of the polymers are listed in Table 15.

TABLE 15
Composition and physicochemical characteristics of the
polydioxanone-based multi-block copolymers

| | | | | | | ppDxx | |
|---|---|---|---|---|---|---|---|
| | | ppCPxxpCyy-block | | | | block | |
| | | Mₙ | | | PCL | Mₙ | |
| | | ppCPxCz | PEG | PEG | length | ppDx | IV |
| Polymer grade | RCP | (g/mol) | MW | content | (g/mol) | (g/mol) | (dl/g) |
| 57CP10C20-D28 | 1502 | 2000 | 1000 | 28.5% | 500 | 2767 | 1.43 |
| 35CP15C20-D24 | 1567 | 2000 | 1500 | 26.3% | 250 | 2437 | 0.63 |
| 50CP15C20-D24 | 1556 | 2000 | 1500 | 37.5% | 250 | 2405 | 0.95 |
| 20CP30C40-D23 | 1557 | 4000 | 3000 | 15% | 500 | 2259 | 0.80 |
| 60CP10C20-D25 | 1565 | 2000 | 1000 | 30% | 500 | 2495 | 0.77 |
| 60CP10C12.5-D22 | 15100 | 1250 | 1000 | 48% | 62 | 2241 | 0.91 |
| 60CP10C16.7-D24 | 15102 | 1670 | 1000 | 36% | 335 | 2401 | 0.89 |

Polymer-only microspheres were prepared by a solvent extraction/evaporation based oil-in-water emulsification process. 5.8 g of polymer dissolved in 52.4 g of dichloromethane (10.0 wt. %) was emulsified in 3.08 kg of ultrapure water containing 4.0 wt. % PVA and 5 wt. % NaCl via membrane emulsification using a membrane with a pore size of 20 gm. The resulting microspheres were collected on a 5 gm membrane filter and washed three times with 250 ml of ultrapure water containing 0.05 wt. % of Tween® 80 and three times with 250 g of ultrapure water. Finally, the microspheres were lyophilised. Particle size measurement and microscopic examination by SEM imaging were carried out following the same procedures as described in Example 3.

The in vitro erosion of non-loaded polymer-only microspheres were measured in 100 mM of phosphate buffer pH 7.4 (90-100 mg of microspheres in 10 ml). The samples were incubated at 37° C. At each sampling point, the microspheres were collected, freeze-dried and weighed.

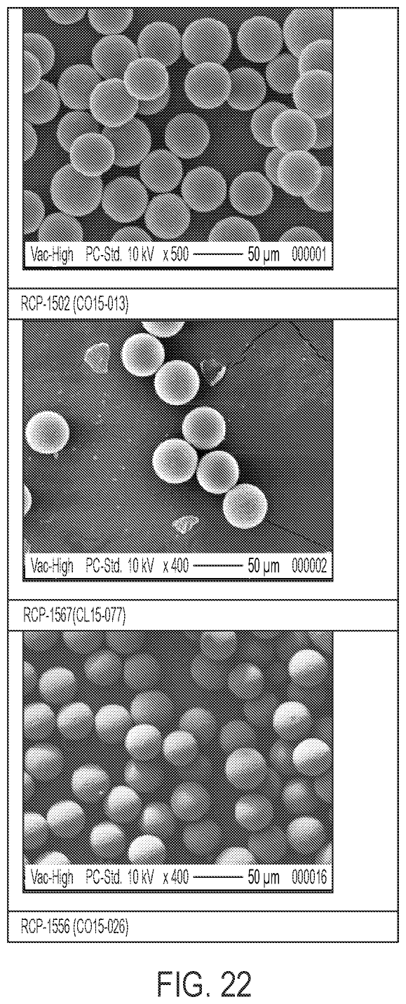

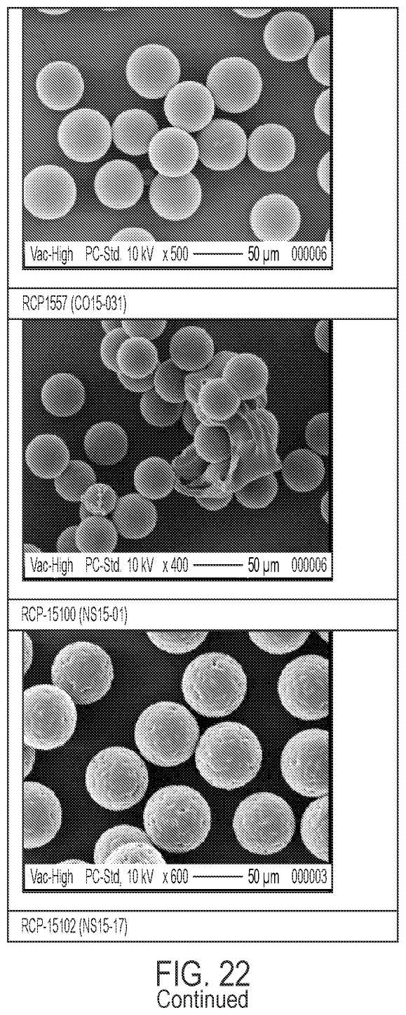

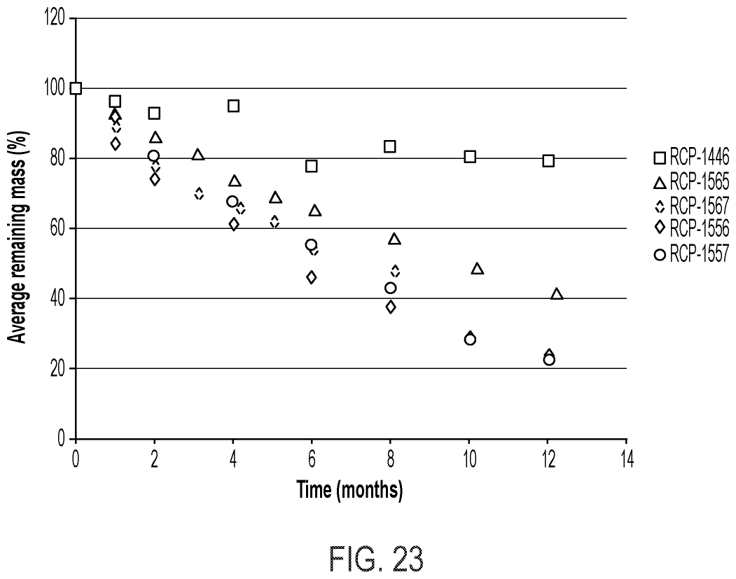

The various dioxanone-based multi-block copolymers were all well processable into microspheres. For all polymers spherical microspheres with a smooth surface morphology (FIG. 22) and an average size varying from 42 to 55 μm were obtained. FIG. 23 shows the effect of PEG molecular weight and PEG content of the hydrophilic block as well as the block ratio on in vitro erosion of D-MBCP-based polymer-only microspheres. Polymer-only microspheres composed of PCLO5 (50CP10C20-LL40) were included as reference material. The erosion rate of all multi-block copolymers composed of poly(dioxanone)-based crystalline blocks was significantly faster as compared to PCL05. After 12 months the remaining mass of polymer-only microspheres composed of 50CP10C20-LL40 was approximately 80%. By replacing the LL40 block by polydioxanone significantly faster eroding polymers were obtained. The remaining mass of polymer-only microspheres composed of 60CP10C20-D25 was around 40% after 12 months. By replacing PEG1000 by PEG1500 or PEG3000 the erosion rate could be further increased. Polymer-only microspheres composed of 30CP15C20-D24, 50CP15C20-D23 and 20CP30C40-D23 exhibited almost linear erosion kinetics with only 20-25% remaining mass after 12 months.

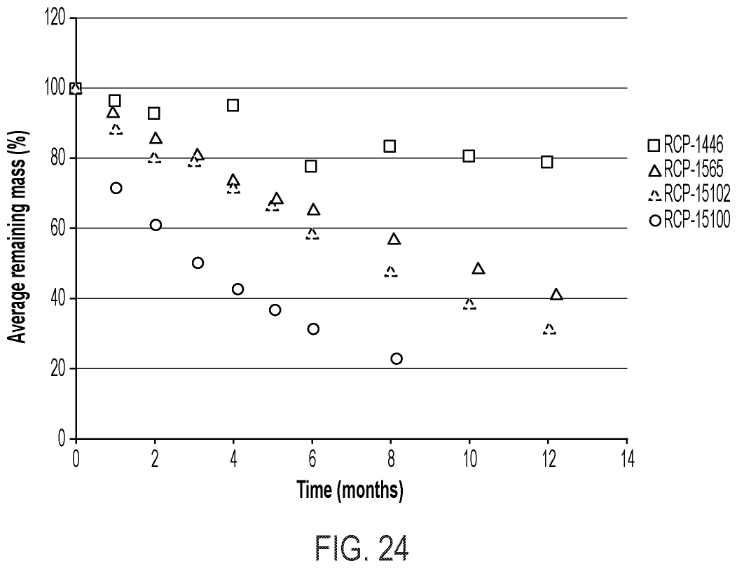

Furthermore, the erosion rate of the overall multi-block copolymers was found to increase significantly with decreasing length of the poly(ϵ-caprolactone) chains of the hydrophilic block (FIG. 24). This is attributed to the higher PEG content (and higher water-swell-ability) of the multi-block copolymers composed of hydrophilic blocks containing shorter poly(ϵ-caprolactone) chains.

### Example 7: Selection of poly(dioxanone)-based Multi-Block Copolymers with the most Optimal Combination of Abicipar Pegol Release and Polymer Erosion Kinetics

For the purpose of screening for poly(dioxanone)-based multi-block copolymers that meet all the requirements for sustained and intraocular delivery of abicipar pegol, the in vitro release (IVR) kinetics of abicipar pegol loaded microspheres and the in vitro erosion (IVE) kinetics of polymer-only microspheres composed of poly(dioxanone)-based multi-block copolymers with different compositions (synthesized as described in Example 3) were compared and IVE/IVR ratios of these polymers were calculated as an arbitrary index for polymer residence time relative to abicipar pegol release duration. Abicipar pegol loaded microspheres with ˜5 wt. % abicipar pegol loading were prepared and characterized as described in Example 5 and evaluated for their abicipar pegol release duration. Polymer-only microspheres were prepared and analysed for their in vitro erosion kinetics as described in Example 6. Table 16 shows in vitro release duration of abicipar pegol microspheres and in vitro erosion duration of polymer-only microspheres composed of different poly(dioxanone)-based multi-block copolymers. All poly(dioxanone)-based multi-block copolymers degraded much faster than the 50CP10C20-LL40 reference material, but only a few polymers provided sustained release of abicipar pegol for >2 months. Abicipar pegol loaded microspheres composed of 57CP10C20-D23 had the best overall profile with the lowest IVE/IVR ratio (3.2) and sustained release of abicipar pegol for 5 months.

TABLE 16
Abicipar pegol in vitro release duration, in vitro polymer erosion
duration and IVE/IVR ratios of various poly(dioxanone)-based
multi-block copolymers.

| | | In vitro | In vitro | |
|---|---|---|---|---|
| | | release | erosion | |
| Multi-block | | duration | duration | IVE/IVR |
| copolymer | Polymer batch | (IVR) | (IVE) ^{a)} | ratio ^{b)} |
| 50CP10C20-LL40 | RCP-1312/ | 4.5 months | 3-4 years | 8-11 |
| | 1446A | | | |
| 57CP10C20-D23 | RCP-1502 | 5 months | 16 months | 3.2 |
| 40CP10C12.5-D23 | RCP-15106 | 3 weeks | 11 months | >10 |
| 40CP10C16.7-D23 | RCP-15108 | 2.3 months | 16 months | 7 |
| 50CP10C14.3-D23 | RCP-15104 | 1.4 months | 12 months | 9 |
| 60CP10C12.5-D22 | RCP-15100 | 1 week | 9 months | >10 |
| 60CP10C16.7-D24 | RCP-15102 | 2.6 months | 15 months | 6 |
| 30CP15C20-D24 | RCP-1567 | 1.8 months | 10 months | 6 |
| 50CP15C20-D24 | RCP-1556 | 1 month | 13 months | 13 |
| 30CP15C30-D23 | RCP-15103 | >2 months ^{c)} | 14 months | <7 |
| 30CP15C50-D23 | RCP-15101 | 2.6 months | 16 months | 6 |
| 40CP15C37.5-D23 | RCP-15110 | >2 months ^{c)} | 18 months | <9 |
| 50CP15C30-D23 | RCP-15109 | >4 months ^{c)} | 18 months | <5 |
| 50CP15C50-D23 | RCP-15107 | 2.8 months | 24 months | 9 |
| 20CP30C40-D23 | RCP-1557 | 2 months | 13 months | 7 |

^{a)} Determined by linear extrapolation of the remaining mass curve to 10% of remaining mass. Each in vitro erosion experiment was performed for at least 8 months.
^{b)} Ratio of the extrapolated in vitro erosion duration and the in vitro abicipar pegol release duration
^{c)} Formulations did not show sustained release of abicipar pegol. They showed a burst release of abicipar pegol and thereafter hardly any release of abicipar pegol (<20%).
^{d)} 50CP10C20-LL40 is shown as reference material.

### Example 8: Effect of Molecular Weight of Polydioxanone Block of 60CP10C20-Dxx on Microsphere Processability, Abicipar Pegol Release and Polymer Erosion Kinetics

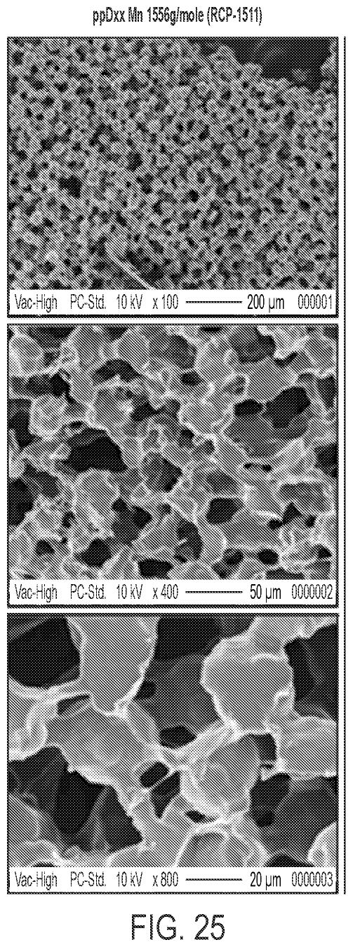

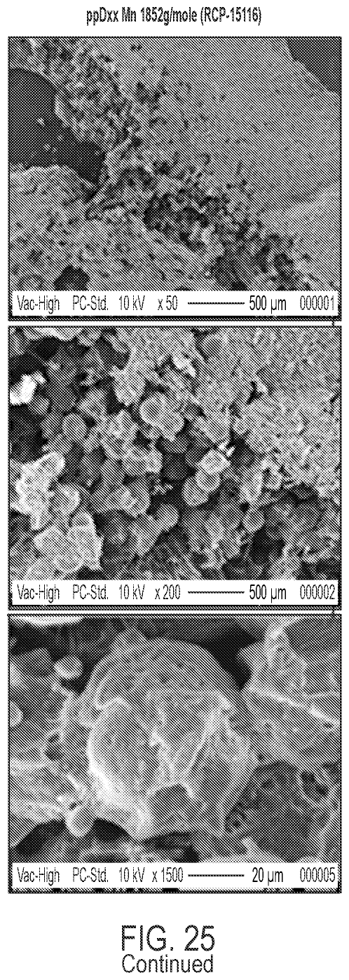

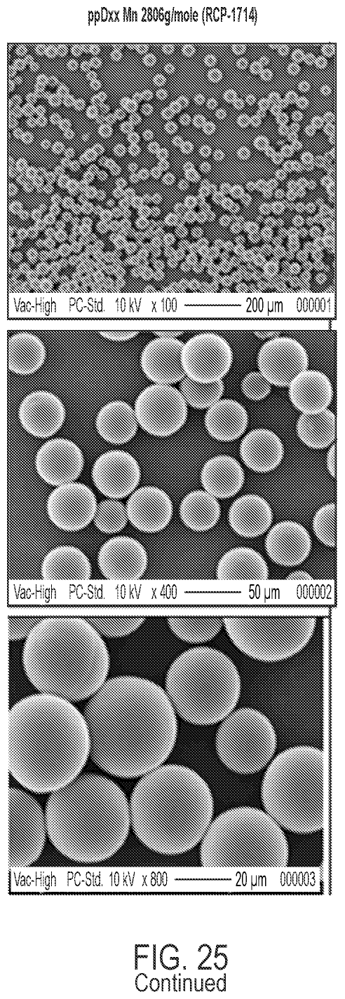

Based on the promising in vitro release kinetics and polymer erosion profile, 60CP10C20-D23 was selected for further optimization of sustained release abicipar pegol microspheres. To further characterize the 60CP10C20-Dxx based abicipar pegol microspheres the effect of Mₙ of the polydioxanone pre-polymer block of 60CP10C20-Dxx on microsphere processability and crystallization of the polydioxanone block was investigated in more detail. 60CP10C20-Dxx multi-block copolymers composed of poly(dioxanone) prepolymer blocks with Mₙ varying from 1556 g/mol to 2806 g/mol (Table 17) were synthesized as described in Example 4. Polymer-only microspheres were prepared and analysed for particle size and microscopic appearance as described in Example 6. Microspheres prepared of RCP1511 (Mₙ D-block 1556 g/mol) and RCP15116 (Mₙ D-block 1852 g/mol) exhibited poor processability (formation of polymer threads, smearing) yielding sticky microspheres that showed severe agglomeration (Table 17, FIG. 25). Microspheres prepared of 60CP10C20-Dxx multi-block copolymers composed of D-blocks with Mₙ exceeding 2200 g/mol (RCP1721, RCP1711, RCP1720 and RCP1714) exhibited excellent processability yielding spherical microspheres with a smooth surface and no visible surface porosity (FIG. 25) and exhibited good powder flowability without any tendency to agglomerate.

Thermal characteristics of the microspheres were analysed by modulated differential scanning calorimetry (m-DSC) using a Q2000 DSC (TA Instruments) as described in Example 4. For the polymers, the melting temperature (Tₘ) and corresponding melting enthalpy (ΔHₘ) of the semi-crystalline poly(p-dioxanone) blocks were determined from the reversed heat flow. For the polymer-only microspheres, Tₘ and ΔHₘ were determined from the total heat flow of the first heating run.

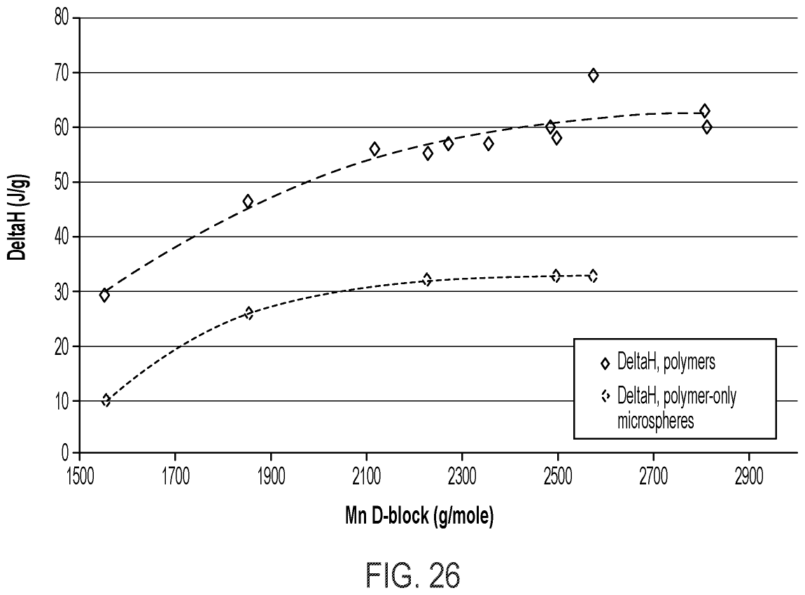

Thermal analysis showed that polymer-only microspheres prepared of 60CP10C20-Dxx composed of poly(dioxanone) pre-polymer blocks with low Mₙ (RCP1511, RCP15116) had a significantly lower melting temperature and melting enthalpy as compared to polymer-only microspheres prepared of 60CP10C20-Dxx multi-block copolymers composed of poly(dioxanone) prepolymer blocks with Mₙ exceeding 2200 g/mol (RCP1721, RCP1711, RCP1720 and RCP1714). At low D-block Mₙ, ΔHₘ increased sharply with D-block Mₙ, whereas at higher D-block Mₙ, ΔHₘ appeared to plateau at a maximum ΔHₘ of around 30-35 J/g (FIG. 26). Clearly, the poor microsphere processability, sticky character and extensive agglomeration observed for polymer-only microspheres prepared of 60CP10C20-Dxx multi-block copolymers with D-blocks of low molecular weight can be attributed to poor crystallization of the polydioxanone pre-polymer block.

TABLE 17
Processability, average particle size (D₅₀) and thermal characteristics
(melting temperature Tₘ and melting enthalpy) of polymer-only
microspheres prepared of 60CP10C20-Dxx multi-block
copolymers composed of poly(dioxanone) blocks of different Mₙ.

| | | Mₙ | | Polymer | | PO-microsphcres | | |
|---|---|---|---|---|---|---|---|---|
| | IV | (g/mol) | | *Tₘ | ΔH* | D₅₀ | *Tₘ | ΔH* |
| RCP | (dl/g) | D-block | Processability | (° C.) | (J/g) | (μm) | (° C.) | (J/g) |
| 1511 | 0.89 | 1556 | Poor processability, sticky | 76.1 | 29.5 | 67.4 | 71.3 | 9.7 |
| | | | microspheres, severe | | | | | |
| | | | agglomeration | | | | | |
| 15116 | 0.86 | 1852 | Poor processability, sticky | 79.3 | 46.5 | 42.5 | 71.1 | 25.9 |
| | | | microspheres, severe | | | | | |
| | | | agglomeration | | | | | |
| 1710 | 0.77 | 2116 | N.D. | 83.2 | 56.1 | — | — | — |
| 1721 | 0.80 | 2226 | Moderate processability | 83.0 | 55.2 | 74.1 | 78.3 | 32.1 |
| 1707 | 0.79 | 2269 | N.D. | 83.9 | 57.0 | — | — | — |
| 1718 | 0.78 | 2356 | N.D. | 86.3 | 56.9 | — | — | — |
| 1719 | 0.89 | 2484 | N.D. | 86.1 | 60.3 | — | — | — |
| 1711 | 0.81 | 2497 | Non sticky microspheres, | 85.7 | 58.0 | 50.9 | 76.2 | 32.9 |
| | | | no agglomeration | | | | | |
| 1720 | 0.82 | 2575 | Non sticky microspheres, | 89.2 | 69.4 | 45.2 | 87.2 | 32.8 |
| | | | no agglomeration | | | | | |
| 1714 | 0.81 | 2806 | Non sticky microspheres, | 88.3 | 63.0 | 49.3 | 87.5 | 35.8 |
| | | | no agglomeration | | | | | |
| 1715 | 0.84 | 2811 | N.D. | 89.1 | 60.0 | — | — | — |

IV is intrinsic viscosity, Mn is number averaged molecular weight
*Tₘ and ΔH of polymers were generated from 2^{nd} heating scan. Tₘ and ΔH of polymer-only microspheres were determined from the total heat flow of the first heating run.

CP10C20-Dxx multi-block copolymers that exhibited good microsphere processability and that yielded non-sticky microspheres without any agglomeration were used for the preparation of abicipar pegol loaded microspheres. Abicipar pegol loaded microspheres with a target abicipar pegol loading of 6.3 wt. % were prepared according to Example 5 using an 167 mg/ml abicipar pegol solution and characterized for particle size, microscopic appearance, abicipar pegol content and in vitro release kinetics as described in Example 5.

Spherical microspheres with a smooth surface and no visible surface porosity were obtained for abicipar pegol loaded microspheres prepared of 60CP10C20-Dxx multi-block copolymers composed of D-blocks of different molecular weight, except for abicipar pegol loaded microspheres composed of RCP1710 (batch MS17-022) with a dioxanone block with Mₙ 2116 g/mol, which exhibited non-spherical and irregularly formed microspheres with large surface pores. RCP1710-based abicipar pegol microspheres also had the lowest abicipar pegol loading, i.e. 2.9 wt. %, representing an encapsulation efficiency of only 46%, and by far the highest burst release (67%) (Table 18). Abicipar pegol loaded microspheres prepared of 60CP10C20-Dxx multi-block copolymers composed of D-blocks with Mₙ>2116 g/mol had significantly lower burst release (4-15%). The extensive particle agglomeration, low abicipar pegol encapsulation efficiency and high burst release of RCP1710-based abicipar pegol microspheres are attributed to poor crystallization of the polydioxanone block of RCP1710 due to its relatively low molecular weight.

TABLE 18
Encapsulation efficiency (EE), actual abicipar pegol loading, particle size
distribution (PSD) and burst release of abicipar pegol loaded microspheres
prepared of multi-block copolymers with different poly(p-dioxanone) Mₙ.

| | | | Abicipar | | Burst | | | |
|---|---|---|---|---|---|---|---|---|
| | Multi-block | | pegol | EE | release | PSD (μm) | | |
| MSP lot | copolymer | RCP # | (wt %) | (%) | (%) | d₁₀ | d₅₀ | d₉₀ |
| MS17-022 | 60CP10C20-D21 | 1710 | 2.9 | 46 | 67 | 40 | 54 | 73 |
| SR17-020 | 60CP10C20-D23 | 1707 | 4.4 | 71 | 6 | 34 | 45 | 60 |
| SR17-030 | 60CP10C20-D24 | 1718 | 4.0 | 64 | 15 | 35 | 45 | 60 |
| SR17-031 | 60CP10C20-D25 | 1719 | 4.6 | 72 | 8 | 36 | 48 | 65 |
| MS17-023 | 60CP10C20-D25 | 1711 | 3.4 | 55 | 9 | 38 | 51 | 69 |
| MS17-031 | 60CP10C20-D26 | 1720 | 3.9 | 63 | 4 | 34 | 45 | 60 |
| MS17-024 | 60CP10C20-D28 | 1714 | 4.3 | 68 | 8 | 33 | 54 | 75 |
| SR17-026 | 60CP10C20-D28 | 1715 | 3.4 | 53 | 8 | 35 | 48 | 64 |

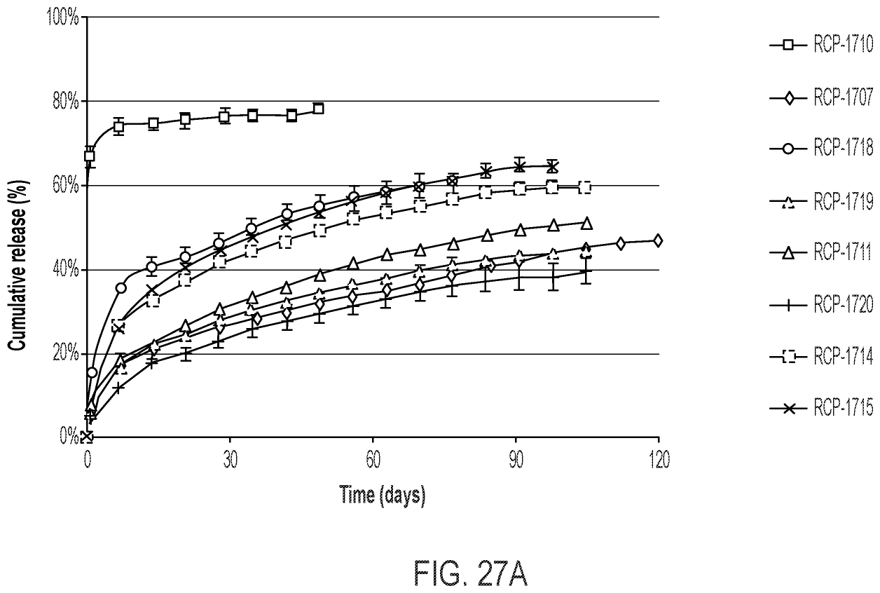

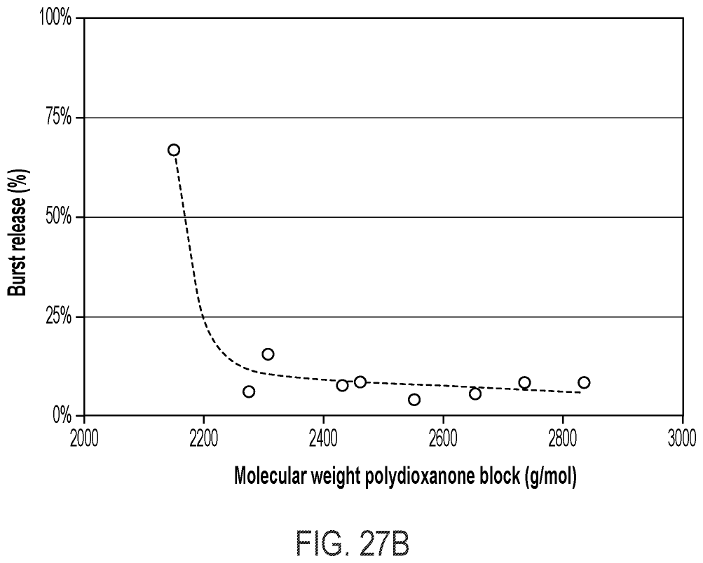

Except for the abicipar pegol microspheres prepared of RCP-1710 (MS17-022) all abicipar pegol-loaded microspheres showed sustained release for at least 3 months (FIG. 27(A)). The abicipar pegol-loaded microspheres based on RCP-1710 (D21) showed hardly any further release after the initial burst release of 67%. Although there were some differences in the initial release rate during the first week, all abicipar pegol microspheres prepared of 60CP10C20-Dxx multi-block copolymers composed of D-blocks with Mₙ>2269 g/mol exhibited similar release kinetics, irrespective of the molecular weight of the D-block. This is further illustrated in FIG. 27(B) which shows that a consistently low burst release can be obtained for abicipar pegol microspheres prepared of 60CP10C20-Dxx multi-block copolymers composed of D-blocks with Mₙ≥2269 g/mol.

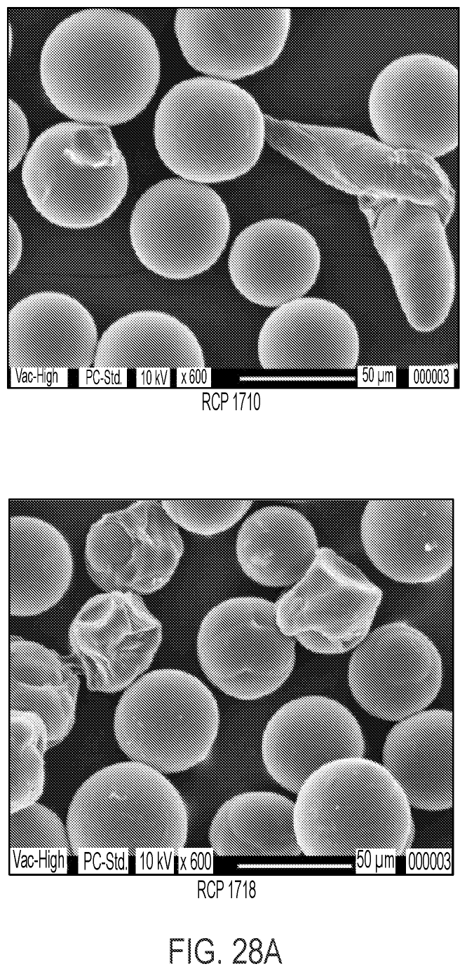

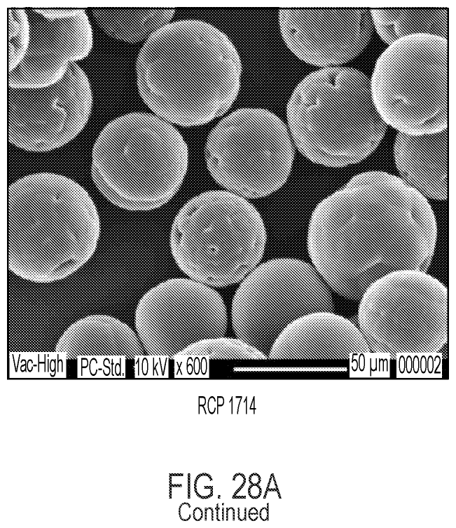

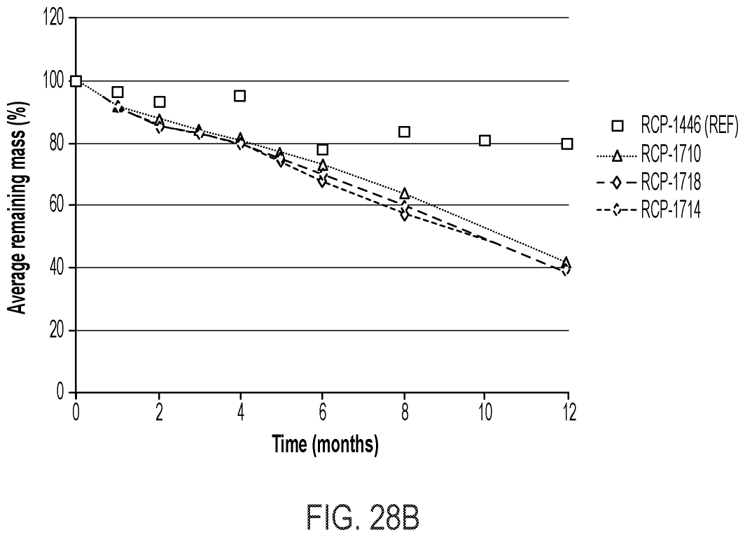

The effect of molecular weight of the poly(dioxanone) block on the in vitro erosion rate was studied in more detail. Polymer-only microspheres were prepared of a selection of 60CP10C20-Dxx multi-block copolymers composed of poly(dioxanone) blocks with molecular weights of 2116 (RCP 1710), 2356 (RCP1718) and 2806 g/mol (RCP1714) and analyzed according to the procedures described in Example 6. Spherical microspheres with a smooth surface, no visible surface porosity and an average particle size of 50 to 55 μm were obtained (FIG. 28A). Thermal analysis of polymer-only microspheres was performed as described in Example 4. The melting temperature increased slightly from 81 to 88° C. with increasing D-block Mₙ whereas the melting enthalpy was relatively constant (24-32 J/g). The molecular weight of the poly(dioxanone) blocks did not impact the in vitro erosion kinetics of microspheres 60CP10C20-Dxx-based microspheres over the range of 2100 to 2800 g/mol (FIG. 28B).

TABLE 19
Thermal properties of polymer-only microsphere batches
used for characterization of in vitro erosion kinetics

| | | | Mₙ | | |
|---|---|---|---|---|---|
| | Multi-block | | D-block | Tₘ | ΔHₘ |
| MSP lot | copolymer | RCP | (g/mol) | (° C.) | (J/g) |
| MS17-068 | 60CP10C20-D21 | 1710 | 2116 | 81 | 29 |
| MS17-069 | 60CP10C20-D24 | 1718 | 2356 | 85 | 24 |
| MS17-070 | 60CP10C20-D28 | 1714 | 2806 | 88 | 32 |

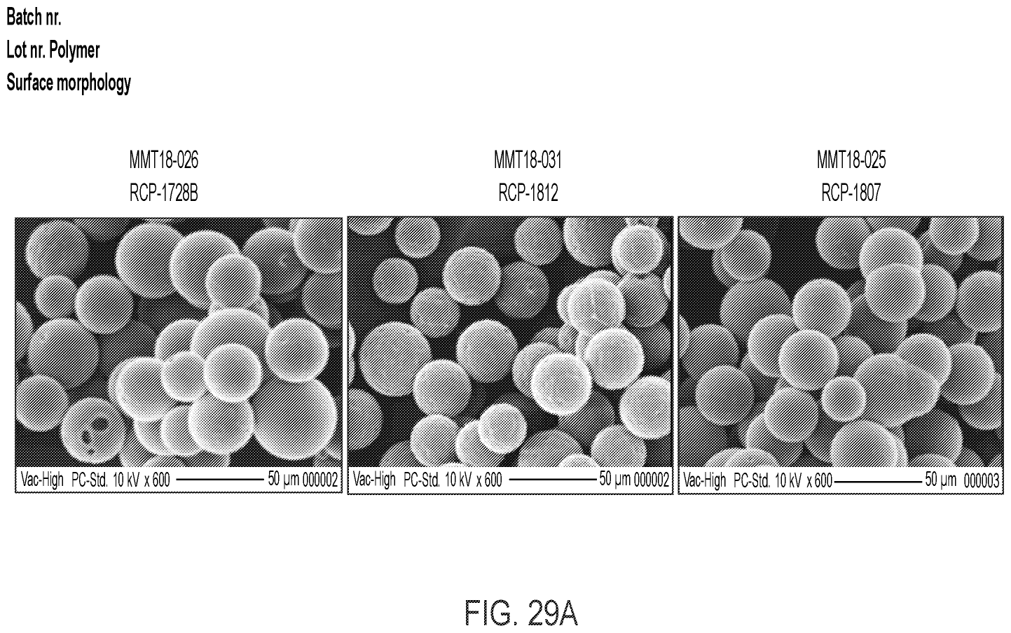

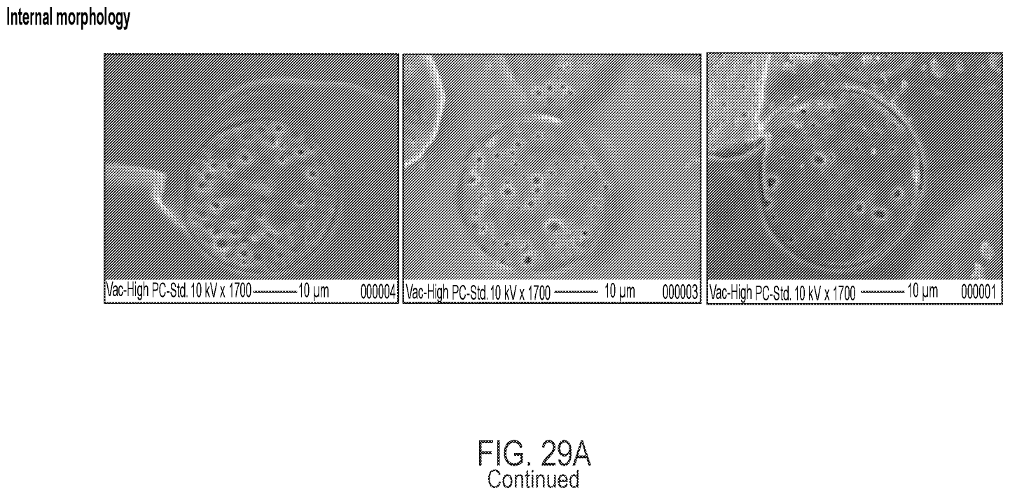

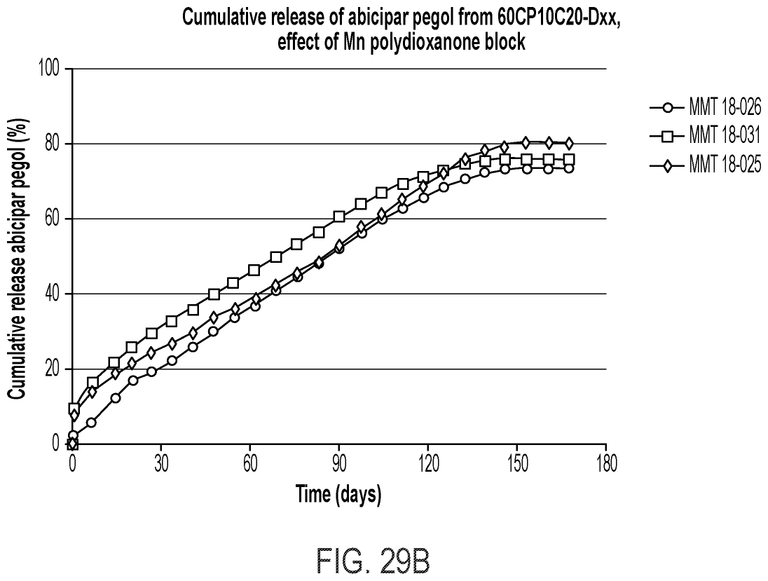

To confirm that Mₙ of the polydioxanone pre-polymer block of 60CP10C20-Dxx multi-block copolymers is not critical for in vitro release kinetics of abicipar pegol microspheres for poly(p-dioxanone) blocks with Mₙ exceeding 2400 g/mol, abicipar pegol loaded microspheres were prepared of 60CP10C20-Dxx multi-block copolymers with polydioxanone blocks with Mₙ 2538, 2887 and 3840 g/mol and characterized for particle size distribution, surface morphology, abicipar pegol content, in vitro release kinetics and thermal characteristics as described above. Irrespective of Mₙ of the polydioxanone pre-polymer block, spherical abicipar pegol loaded microspheres with a smooth surface morphology and an average size of 45-50 gm were obtained. The lyophilized microspheres were free flowing and did not show any tendency to agglomerate (FIG. 29A). Abicipar pegol content varied from 4.9 to 5.5%, representing encapsulation efficiencies of 77 to 81% (Table 20). Despite some variations in Tₘ (82-93° C.) and ΔHₘ (24-31 J/g), the in vitro release of abicipar pegol from the microspheres was hardly affected by Mₙ of the poly(dioxanone) blocks of the 60CP10C20-Dxx multi-block copolymers for poly(p-dioxanone) pre-polymer blocks with Mₙ varying from 2538 to 3840 g/mol (FIG. 29B).

TABLE 20
Characteristics of abicipar pegol microspheres prepared of
60CP10C20-Dxx copolymers with poly(p-dioxanone) pre-polymer blocks of
different Mₙ.

| Polymer | | | | Abicipar pegol microspheres | | | | | |
|---|---|---|---|---|---|---|---|---|---|
| | Mₙ | | | | | | | abicipar | |
| | D-block | Tₘ | ΔHₘ | | d₅₀ | Tₘ | ΔHₘ | pegol | |
| RCP | (g/mol) | (° C.) | (J/g) | MSP lot # | (μm) | (° C.) ^{a)} | (J/g) ^{a)} | content | EE |
| 1728B | 2538 | 87 | 54 | MMT18-026 | 48 | 82 | 26 | 5.5% | 80% |
| 1812 | 2887 | 89 | 69 | MMT18-031 | 44 | 88 | 24 | 4.9% | 77% |
| 1807 | 3840 | 95 | 70 | MMT18-025 | 46 | 93 | 31 | 5.1% | 81% |

### Example 9: PCD21-Based Abicipar Pegol Microspheres with Optimized Release Kinetics (Low Burst Release)

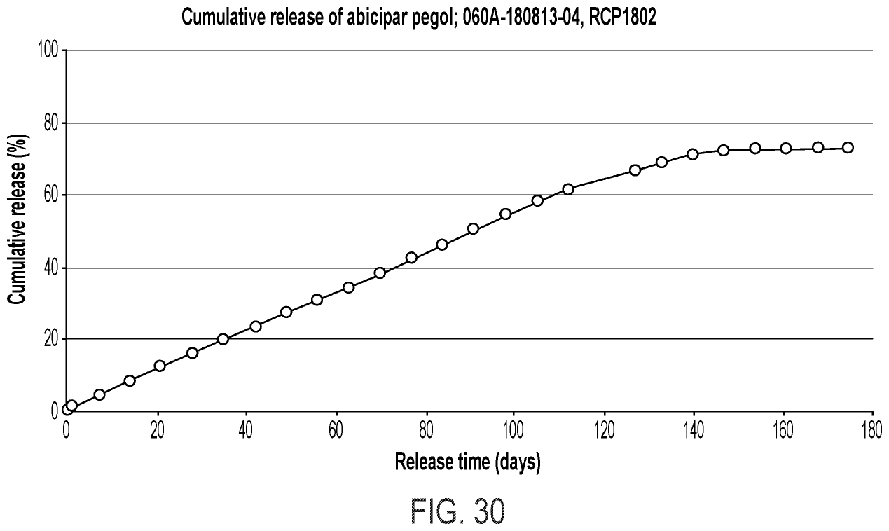

Abicipar pegol loaded microspheres were prepared of PCD21 at a scale of 2.5 g using a W1/O/W2 water-in-oil-in-water double emulsion-based membrane emulsification process similar as described in Example 5. Abicipar pegol was dissolved in PBS to a concentration of 170 mg/g and PCD21 was dissolved in dichloromethane to a concentration of 10 wt. %. The microparticles were analyzed for microscopic surface morphology (SEM), particle size, abicipar pegol load and in vitro release kinetics according to the methods described in Example 3. The microspheres had a smooth surface without any pores and an average particle size (D₅₀) of 77.4 μm. The abicipar pegol content was 5.14 wt. % representing an encapsulation efficiency of 81.3%. The microspheres released abicipar pegol continuously for 5 months according to linear release kinetics and without any significant burst release as shown in FIG. 30.

### Example 10: Manufacturing and Characterization of PCD21-Based Abicipar Pegol Microspheres at a Scale of 25g

Abicipar pegol loaded microspheres were prepared of PCD21 at a scale of 25 g using a W1/O/W2 water-in-oil-in-water double emulsion-based membrane emulsification process similar as described in Example 9. 2.8 g of abicipar pegol was dissolved in PBS to a concentration of 170 mg/g and 30 g of PCD21 was dissolved in dichloromethane to a concentration of 10 wt. %. The polymer solution and protein solution were subsequently pumped at constant flow rates into and homogenized using an in-line high shear mixer.

The primary emulsion was then immediately emulsified with an aqueous 0.4% w/v PVA solution using a membrane emulsification unit thereby forming a secondary emulsion. Abicipar pegol microspheres were hardened following DCM extraction and evaporation, further concentrated using a Nutsche filter dryer and washed with WFI. The semi-dry microsphere powder was cooled down to −10° C. and further vacuum-dried.

Abicipar pegol microspheres were analysed for appearance, surface morphology, particle size, abicipar pegol content, and in vitro release kinetics as described in Example 3. Abicipar pegol purity was determined by UP-SEC. UP-SEC analysis was conducted using a Waters ACQUITY UPLC Protein BEH SEC Column and a fluorescence detector (λₑₓ =280 nm, λₘ=350 nm). The potency of abicipar pegol was measured by a sandwich ELISA technique.

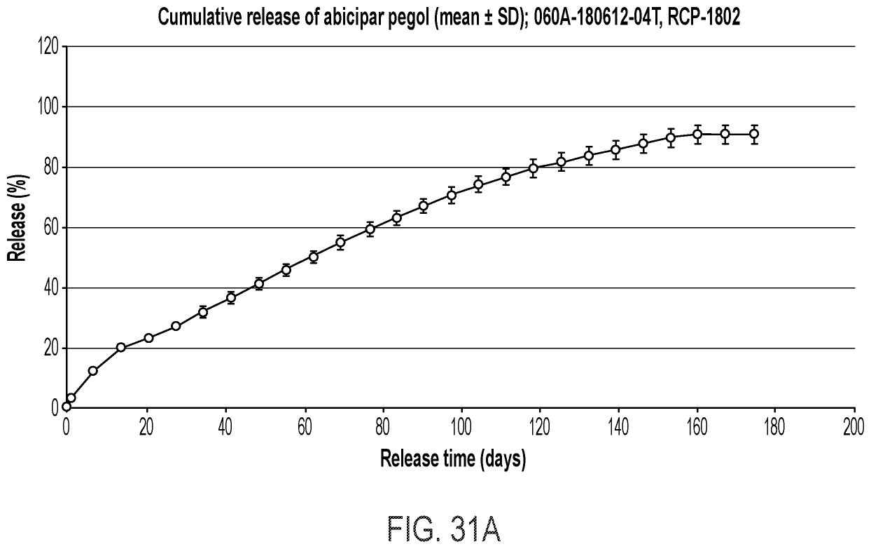

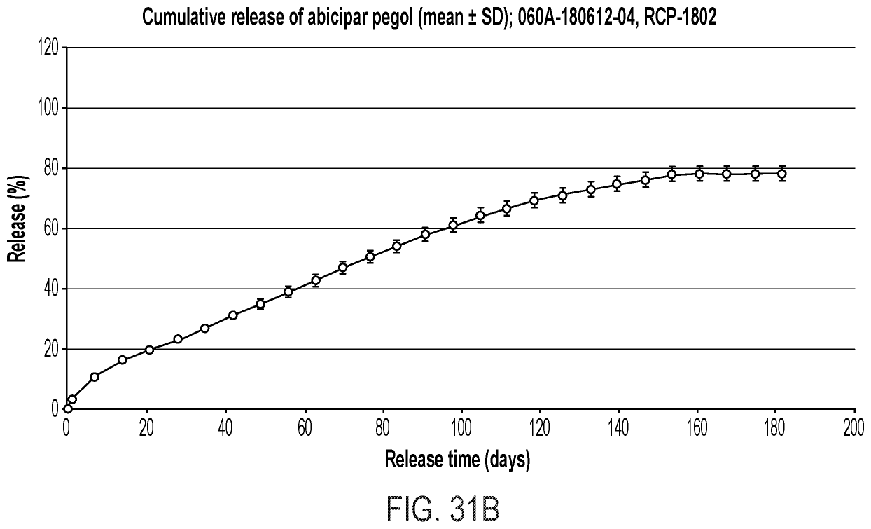

The microspheres were spherical and had a smooth surface without any pores and an average particle size (D₅₀) of 77.0 μm. The abicipar pegol content was 5.6 wt. %. The potency of encapsulated abicipar pegol as analysed by the sandwich ELISA method was 107%. The purity of encapsulated abicipar pegol was found to be 99.0% as determined with SEC-UPLC. The microspheres released abicipar pegol continuously for 5 months. The concentrations of total and intact abicipar pegol as measured at each time point are shown in Table 21. The purity of released abicipar pegol was on average 88% (range 78-95%). Cumulative release profiles of total and intact abicipar pegol are shown in FIGS. 31A-B.

TABLE 21
Concentrations (in μg/ml) of total and intact abicipar pegol and
purity of released abicipar pegol per time point (batch nr 060A-180612-04;
purity as determined by UP-SEC with FLR detection.

| | abicipar pegol | | |
|---|---|---|---|
| | conc (μg/ml) | | |
| Time | Total | Intact | Purity |
| (days) | (μg/ml) | (μg/ml) | (%) |
| 1 | 17.5 | 16.6 | 94.5 |
| 7 | 53.8 | 46.4 | 86.2 |
| 14 | 47.9 | 39.7 | 83.0 |
| 21 | 25.2 | 22.1 | 87.6 |
| 28 | 27.9 | 22.6 | 81.1 |
| 35 | 31.7 | 26.1 | 82.3 |
| 42 | 29.8 | 24.7 | 82.9 |
| 49 | 28.6 | 24.0 | 83.9 |
| 56 | 28.7 | 24.5 | 85.4 |
| 63 | 30.8 | 26.7 | 86.6 |
| 70 | 29.0 | 25.4 | 87.6 |
| 77 | 26.5 | 23.4 | 88.6 |
| 84 | 25.4 | 22.6 | 89.1 |
| 91 | 25.5 | 22.9 | 89.9 |
| 98 | 23.8 | 21.8 | 91.5 |
| 105 | 22.3 | 20.5 | 92.0 |
| 112 | 17.7 | 16.4 | 92.8 |
| 119 | 17.3 | 16.2 | 93.4 |
| 126 | 13.9 | 12.9 | 92.7 |
| 133 | 13.2 | 12.2 | 93.0 |
| 140 | 12.6 | 11.4 | 90.6 |
| 147 | 12.3 | 9.6 | 78.0 |
| 154 | 12.9 | 9.4 | N.D.* |
| 161 | 7.9 | 3.3 | N.D.* |

*could not be determined due to too low concentration of abicipar pegol

### Example 11: Reproducibility of PCD21-Based Abicipar Pegol Microspheres Manufacturing (at 25 g Batch Size)

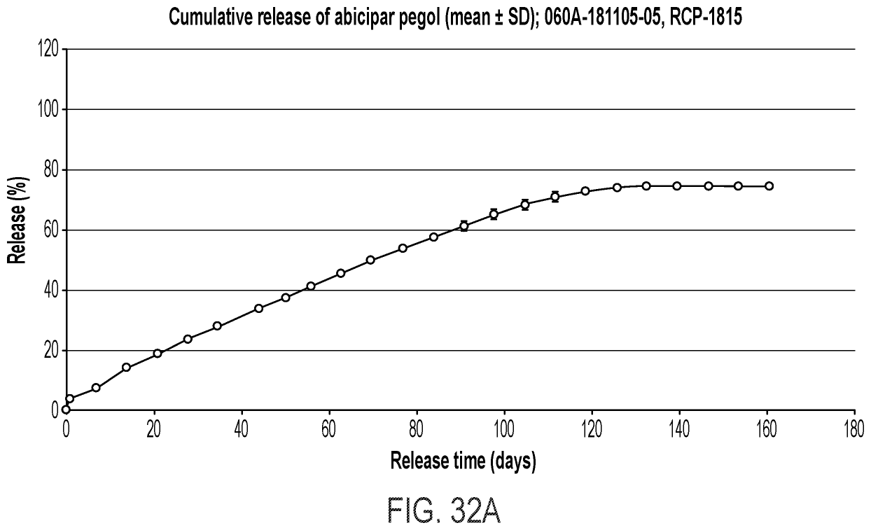

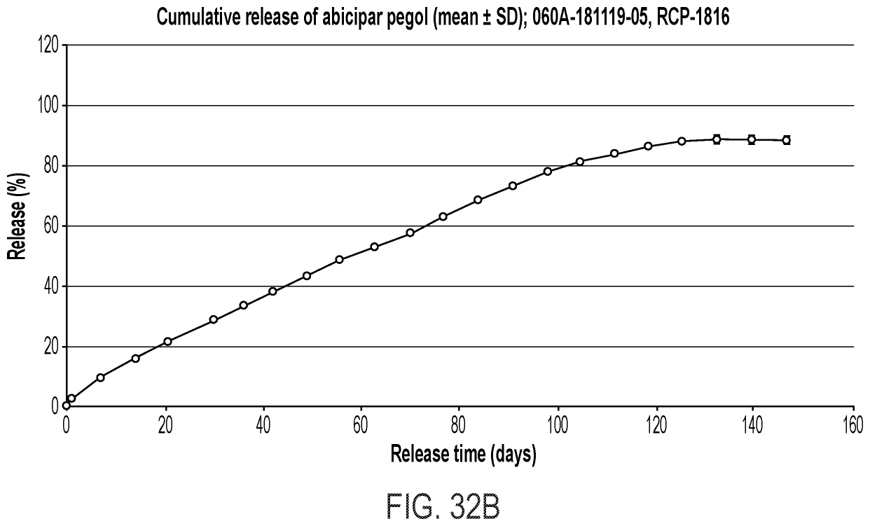

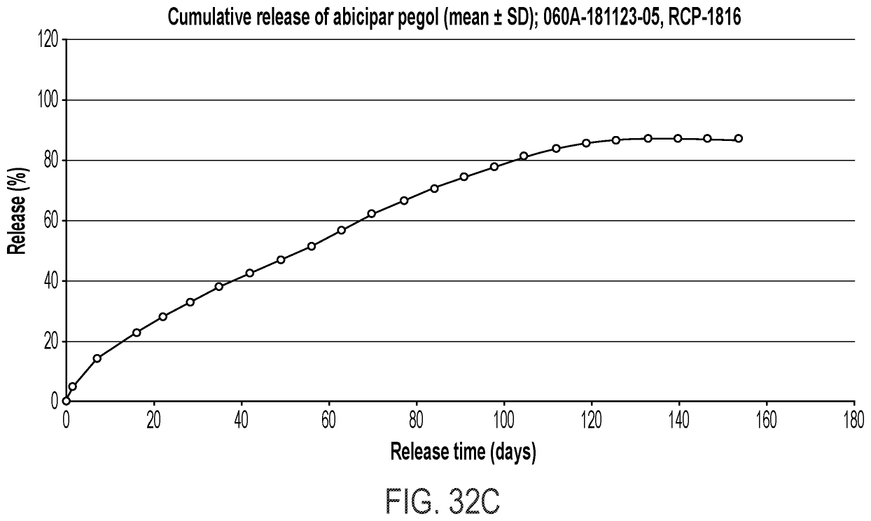

Three 25 g batches of PCD21-based abicipar pegol loaded microspheres were manufactured as described in Example 10. 060A-181105-05 was manufactured of PCD21 polymer batch RCP-1815 whereas. 060A-181119-05 and 060A-181123-05 were manufactured of PCD21 polymer batch RCP-1816. The batches were analysed according the analytical methods described in Examples 3 and 10. The results obtained for the three batches were very similar (Table 22). FIGS. 32A-C shows the cumulative release kinetics of abicipar pegol of the three individual batches. The potency of encapsulated abicipar pegol as analysed by the sandwich ELISA method was 95%.

TABLE 22
Characteristics of PCD21-based abicipar pegol microsphere
batches manufactured at a batch size of 25 g.

| | Analytical | | | | | | |
|---|---|---|---|---|---|---|---|
| Test | method | 060A-181105-05 | | 060A-181119-05 | | 060A-181123-05 | |
| Appearance | Microscopic | Spherical | | Spherical | | Spherical | |
| | examination (SEM) | particles | | particles | | particles | |
| Average | Laser diffraction | 74 | μm | 61 | μm | 60 | μm |
| particle size | | | | | | | |
| (D₅₀) | | | | | | | |
| Abicipar | Extraction & | 6.1 | wt. % | 5.3 | wt. % | 5.3 | wt. % |

| | | | | |
|---|---|---|---|---|
| pegol content | SEC-UPLC | | | |
| Purity | Extraction & | 97.7% | 98.6% | 98% |
| | SEC-UPLC | | | |
| Impurity | Extraction & | 2.3% | 1.4% | 2.0% |
| profile | SEC-UPLC | | | |

1. A pharmaceutical composition for the treatment of an ocular disorder in a patient in need thereof, comprising (a) a biologically active compound; and (b) a biodegradable, semi-crystalline, phase separated, thermoplastic poly(ether ester) multi-block copolymer;
wherein said biologically active compound is abicipar pegol, wherein said multi-block copolymer comprises (i) an amorphous hydrolysable pre-polymer (A) segment having the following formula: (R′R²ₙR³)_{q}; and (ii) a semi crystalline hydrolysable pre polymer (B) segment having the following formula: (R⁴ₚR⁵R⁶ₚ); arranged according to Formula (PEE-MBCP):
[(R¹R²ₙR³)_{q}]r[(R⁴ₚR⁵R⁶ₚ)]ₛ (Formula PEE-MBCP)
wherein each segment is linked by a 1,4 butanediisocyanate chain extender,
wherein said segments are randomly distributed over the polymer chain;
wherein
R¹ and R³ are each 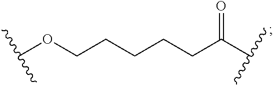 [SMILES: *OCCCCCC(*)=O]
R² is 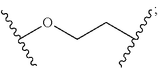 [SMILES: *CCO*]
R⁴ and R⁶ are each 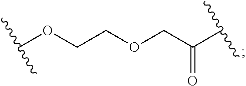 [SMILES: *OCCOCC(*)=O]
R⁵ is 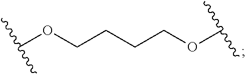
wherein
n, being the number of repeating R² moieties, is about 22 to about 23;
p, being the number of repeating R⁴ and R⁶ moieties, is about 11.5;
q, being the molecular weight of the (R²R²ₙR³) block, is about 2000 g/mol;
r, being the weight fraction of pre-polymer (A) segment relative to the total amount of pre-polymer (A) and (B), is about 60%; and
s, being the weight fraction of pre-polymer (B) segment relative to the total amount of pre-polymer (A) and (B), is about 40%;
wherein said biologically active compound is encapsulated in a matrix comprising said multi-block copolymer; wherein said multi-block copolymer has a T_{g} of 37° C. or less and a Tₘ of 50-110° C. under physiological conditions, and wherein said multi-block copolymer has an intrinsic viscosity of about 0.8 dl/g.

2. The pharmaceutical composition according to claim 1, wherein said composition is in the form of a plurality of polymeric microspheres that are each not less than about 20 μm in diameter.

3. The pharmaceutical composition according to claim 2, wherein said polymeric microspheres are at least 20 μm in diameter.

4. The pharmaceutical composition according to claim 2 or 3, wherein the plurality of polymeric microspheres comprise about 4% to about 6% w/w of said biologically active compound.

5. The pharmaceutical composition according to claim 4, wherein the plurality of polymeric microspheres comprise about 4% w/w of said biologically active compound.

6. The pharmaceutical composition according to claim 4, wherein the plurality of polymeric microspheres comprise about 5% w/w of said biologically active compound.

7. The pharmaceutical composition according to claim 4, wherein the plurality of polymeric microspheres comprise about 6% w/w of said biologically active compound.

8. A pharmaceutical composition for the treatment of an ocular disorder in a patient in need thereof, comprising (a) a biologically active compound; and (b) a biodegradable, semi-crystalline, phase separated, thermoplastic poly(ether ester) multi-block copolymer;
wherein said biologically active compound is abicipar pegol, wherein said multi-block copolymer comprises (i) an amorphous hydrolysable pre-polymer (A) segment having the following formula: (R¹R²ₙR³)_{q}; and (ii) a semi crystalline hydrolysable pre polymer (B) segment having the following formula: (R⁴ₚR⁵R⁶ₚ); arranged according to Formula (PEE-MBCP):
[(R¹R²ₙR³)_{q}]ᵣ[(R⁴ₚR⁶R⁶ₚ)]s (Formula PEE-MBCP)
wherein each segment is linked by a 1,4 butanediisocyanate chain extender,
wherein said segments are randomly distributed over the polymer chain;
wherein
R¹ and R³ are each 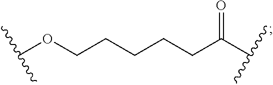 [SMILES: *OCCCCCC(*)=O]
R² is 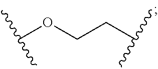 [SMILES: *CCO*]
R⁴ and R⁶ are each 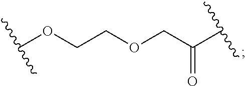 [SMILES: *OCCOCC(*)=O]
R⁵ is 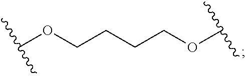
wherein
n, being the number of repeating R² moieties, is about 20 to about 25;
p, being the number of repeating R⁴ and R⁶ moieties, is about 10 to about 13.5;
q, being the molecular weight of the (R¹R²ₙR³) block, is about 1800 to about 2200 g/mol; and
the ratio r/s is 1.1-2.0 wherein r is the weight fraction of pre-polymer (A) segment and s is the weight fraction of pre-polymer (B) segment, relative to the total amount of pre polymer (A) and (B); and
wherein said biologically active compound is encapsulated in a matrix comprising said multi-block copolymer; wherein said multi-block copolymer has a T_{g} of 37° C. or less and a Tₘ of 50-110° C. under physiological conditions.

9. The pharmaceutical composition according to claim 8, wherein said composition is in the form of a plurality of polymeric microspheres that are each not less than about 20 μm in diameter.

10. The pharmaceutical composition according to claim 9, wherein said polymeric microspheres are at least 20 μm in diameter.

11. The pharmaceutical composition according to claim 9 or 10, wherein the plurality of polymeric microspheres comprise about 4% to about 6% w/w of said biologically active compound.

12. The pharmaceutical composition according to claim 11, wherein the plurality of polymeric microspheres comprise about 4% w/w of said biologically active compound.

13. The pharmaceutical composition according to claim 11, wherein the plurality of polymeric microspheres comprise about 5% w/w of said biologically active compound.

14. The pharmaceutical composition according to claim 11, wherein the plurality of polymeric microspheres comprise about 6% w/w of said biologically active compound.

15. A biodegradable, semi-crystalline, phase separated, thermoplastic poly(ether ester) multi-block copolymer comprising (i) an amorphous hydrolysable pre-polymer (A) segment having the following formula: (R¹R²ₙR³)_{q}; and (ii) a semi crystalline hydrolysable pre polymer (B) segment having the following formula: (R⁴ₚR⁵R⁶ₚ); arranged according to Formula (PEE-MBCP):
[(R¹R²ₙR³)_{q}]ᵣ[(R⁴ₚR⁵R⁶ₚ)]s (Formula PEE-MBCP)
wherein each segment is linked by a 1,4 butanediisocyanate chain extender, wherein said segments are randomly distributed over the polymer chain;
wherein
R¹ and R³ are each 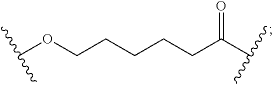 [SMILES: *OCCCCCC(*)=O]
R² is 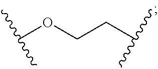 [SMILES: *CCO*]
R⁴ and R⁶ are each 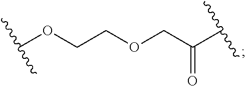 [SMILES: *OCCOCC(*)=O]
R⁵ is 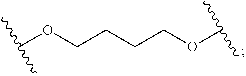
wherein
n, being the number of repeating R² moieties, is about 22 to about 23;
p, being the number of repeating R⁴ and R⁶ moieties, is about 11.5;
q, being the molecular weight of the (R¹R²ₙR³) block, is about 2000 g/mol;
r, being the weight fraction of pre-polymer (A) segment relative to the total amount of pre-polymer (A) and (B), is about 60%; and
s, being the weight fraction of pre-polymer (B) segment relative to the total amount of pre-polymer (A) and (B), is about 40%;
wherein said multi-block copolymer has a T_{g} of 37° C. or less and a Tₘ of 50-110° C. under physiological conditions, and wherein said multi-block copolymer has an intrinsic viscosity of about 0.8 dl/g.
